(19) United States
(12) Patent Application Publication
(10) Pub. No.: US 2024/0076353 A1
(43) Pub. Date: Mar. 7, 2024

(54) Agonistic Interleiukin 15 Complexes and Uses Thereof

(71) Applicant: Suzhou Forlong Biotechnology Co., Ltd., Jiangsu (CN)

(72) Inventors: En Ji, Jiangsu (CN); Chunyu Wang, Jiangsu (CN)

(21) Appl. No.: 18/457,427

(22) Filed: Aug. 29, 2023

(30) Foreign Application Priority Data
Aug. 31, 2022 (WO) PCT/CN2022/116012

(51) Int. Cl. C07K 14/715; A61K 47/64

(52) U.S. Cl. CPC C07K 14/7155; A61K 47/6425; C07K 2319/30; C07K 2319/70

(57) ABSTRACT

The present disclosure provides agonistic IL-15 complexes that have therapeutic use, and methods for making such proteins. The present disclosure additionally provides methods of treating disease in a subject in need thereof by administering such agonistic IL-15 complexes.

### CROSS-REFERENCE TO RELATED APPLICATION

This application claims priority to International Application No. PCT/CN2022/116012, filed Aug. 31, 2022, the disclosure of which is incorporated herein by reference in its entirety.

### REFERENCE TO SEQUENCE LISTING SUBMITTED ELECTRONICALLY

This application contains a sequence listing with a file name “069777.11026-6US1_sequence_listing.xml” and a creation date of Aug. 17, 2023, and having a size of 10 kb. The sequence listing is part of the specification and is herein incorporated by reference in its entirety.

### FIELD

The present disclosure generally relates to compositions and methods comprising interleukin 15 (IL-15) agonists, such as agonistic IL-15 complexes, which agonistic IL-15 complexes can be used, for instance, in methods of treating a disease in a subject in need thereof.

### BACKGROUND

The cytokine interleukin-15 (IL-15) is a member of the four alpha-helix bundle family of lymphokines produced by many cells in the body. IL-15 plays a pivotal role in modulating the activity of both the innate and adaptive immune system, e.g., maintenance of the memory T-cell response to invading pathogens, inhibition of apoptosis, activation of dendritic cells, and induction of Natural Killer (NK) cell proliferation and cytotoxic activity.

The IL-15 receptor consists of three polypeptides, the type-specific IL-15 receptor alpha (“IL-15Ra”), the IL-2/IL-15 receptor beta (or CD122) (“3”), and the common gamma chain (or CD132) (“γ”) that is shared by multiple cytokine receptors. The IL-15Ra is thought to be expressed by a wide variety of cell types, but not necessarily in conjunction with β and γ. IL-15 signaling has been shown to occur through the heterodimeric complex of IL-15Ra, β, and γ; through the heterodimeric complex of β and γ, or through a subunit, IL-15RX, found on mast cells (Ikemizu, Shinji et al. “IL-2 and IL-15 signaling complexes: different but the same.” Nature immunology vol. 13, 12 (2012): 1141-2, PMID: 23160210; Briukhovetska, Daria et al. “Interleukins in cancer: from biology to therapy.” Nature reviews. Cancer vol. 21, 8 (2021): 481-499, PMID: 3408378).

IL-15 is a soluble protein, but endogenous IL-15 is not readily detectable in serum or body fluids—instead, it occurs predominantly as a membrane-bound form that is expressed or acquired by several types of accessory cells (Fehniger, T A, and M A Caligiuri. “Interleukin 15: biology and relevance to human disease.” Blood vol. 97, 1 (2001): 14-32, PMID: 11133738). For instance, although IL-15 mRNA is detected in cells of both hematopoietic and non-hematopoietic lineage, T cells do not produce IL-15. Instead, IL-15 binds to the IL-15Ra, forming cell-surface complexes on T cells. IL-15 specifically binds to the IL-15Ra with high affinity via the “sushi domain” in exon 2 of the extracellular domain of the receptor (Budagian, Vadim et al. “IL-15/IL-15 receptor biology: a guided tour through an expanding universe.” Cytokine & growth factor reviews vol. 17, 4 (2006): 259-80, PMID: 16815076). After trans-endosomal recycling and migration back to the cell surface, these IL-15 complexes acquire the property to activate bystander cells expressing the IL-15R βγ low-affinity receptor complex, inducing IL-15-mediated signaling via the Jak/Stat pathway. A naturally occurring soluble form of IL-15Ra (“sIL-15Ra”), which is cleaved at a cleavage site in the extracellular domain immediately distal to the transmembrane domain of the receptor has been observed (Lukic, M L et al. “Lack of the mediators of innate immunity attenuate the development of autoimmune diabetes in mice.” Journal of autoimmunity vol. 21, 3 (2003): 239-46, PMID: 14599848). Tumor necrosis factor-alpha-converting enzyme (TACE/ADAM17) has been implicated as a protease involved in this process.

Based on its multifaceted role in the immune system, various therapies designed to modulate IL-15-mediated function have been explored. For example, the administration of exogenous IL-15 can enhance the immune function of patients infected with human immunodeficiency virus (HIV). Despite the amount of progress made in understanding the function of IL-15, many challenges remain in IL-15 based therapies, as further discussed infra. As such, there is strong interest in the further development of new IL-15 based therapeutics.

### SUMMARY OF THE INVENTION

The present disclosure generally relates to a fusion protein comprising an interleukin-15 (IL-15) receptor α sushi domain fused to an Fc monomer, wherein the IL-15 receptor α sushi domain comprises the amino acid sequence of SEQ ID NO: 5 and the Fc monomer comprises the amino acid sequence of SEQ ID NO: 6. In some aspects, the carboxyl-terminus of the IL-15 receptor α sushi domain is fused to the amino-terminus of the Fc monomer via a linker having the amino acid sequence of GGGGS (SEQ ID NO: 7). In some aspects, the linker consists of the amino acid sequence of SEQ ID NO: 7 or SEQ ID NO: 8. In some aspects, the fusion protein comprises the amino acid sequence of SEQ ID NO: 1. In some aspects, the fusion protein comprises the amino acid sequence of SEQ ID NO: 2.

Furthermore, the present disclosure generally relates to a fusion protein consisting of the amino acid sequence of SEQ ID NO: 1. Moreover, the present disclosure generally relates to a fusion protein consisting of the amino acid sequence of SEQ ID NO: 2. Additionally, the present disclosure generally relates to a protein complex comprising a fusion protein as described herein and an IL-15. In some aspects, the IL-15 comprises the amino acid sequence of SEQ ID NO: 3. In some aspects, the IL-15 consists of the amino acid sequence of SEQ ID NO: 3. In some aspects, the IL-15 comprises the amino acid sequence of SEQ ID NO: 4. In some aspects, the IL-15 consists of the amino acid sequence of SEQ ID NO: 4.

Moreover, the present disclosure generally relates to a nucleic acid molecule, such as a vector, encoding the fusion protein as described herein. Furthermore, the present disclosure generally relates to a host cell comprising a nucleic acid molecule, such as a vector, encoding the fusion protein as described herein. Additionally, the present disclosure generally relates to a method of producing a fusion protein as described herein, comprising culturing a host cell as described herein under conditions sufficient to express the fusion protein, and isolating the fusion protein. Moreover, the present disclosure generally relates to one or more nucleic acid molecules, such as one or more vectors, encoding a fusion protein and an TL-15 of the protein complex as described herein. Furthermore, the present disclosure generally relates to one or more host cells comprising one or more nucleic acid molecules, such as one or more vectors, encoding a fusion protein and an IL-15 of the protein complex as described herein. Additionally, the present disclosure generally relates to a method of producing a protein complex as described herein, comprising culturing one or more host cells as described herein under conditions sufficient to express the fusion protein and the IL15, isolating the fusion protein and IL15, and optionally assembling the protein complex by combining the isolated fusion protein and IL15 in vitro.

Furthermore, the present disclosure generally relates to a pharmaceutical composition comprising a fusion protein as described herein, a protein complex as described herein, a nucleic acid molecule as described herein, one or more nucleic acid molecules as described herein, a host cell as described herein, or one or more host cells as described herein, and a pharmaceutically acceptable excipient, diluent, or carrier. In some aspects, the pharmaceutical composition comprises a protein complex as described herein.

Moreover, the present disclosure generally relates to a method of treating a disease in a subject in need thereof, comprising administering to the subject an effective amount of a pharmaceutical composition as described herein. In some aspects, the method further comprises administering to the subject another therapeutically active agent. In some aspects, the other therapeutically active agent comprises a small molecule compound, a targeted chemotherapeutic agent, a radiation therapy agent, or an antibody or an antigen binding fragment thereof. In some aspects, the other therapeutically active agent comprises an antibody or an antigen binding fragment thereof that binds specifically to CD20, PD1, PDL1, Her2, EGFR or c-MET. In some aspects, the other therapeutically active agent comprises an anti-PD-1 antibody or an antigen binding fragment thereof. In some aspects, the disease is a tumor. In some aspects, the tumor is a solid tumor. In some aspects, the disease is a pathogen infection, such as a bacterial infection and/or a viral infection. In some aspects, the disease is an autoimmune disease. In some aspects, the disease is an inflammatory disease. In some aspects, the disease is a neurodegenerative disease.

Furthermore, the present disclosure generally relates to a method of treating a tumor, comprising administering to a subject in need thereof an effective amount of a pharmaceutical composition as described herein. In some aspects, the tumor is a solid tumor, such as a carcinoma, a sarcoma, or a melanoma. In some aspects, the solid tumor is selected from the group consisting of a bladder cancer, a lung cancer, a breast cancer, a colorectal cancer, a brain tumor, a prostate cancer, a melanomas, a merkel cell carcinoma, a head and neck cancer, a small bowel cancer, a squamous cell carcinoma, metastatic solid tumor or a cervical cancer. In some aspects, the tumor is a bladder cancer. In some aspects, the tumor is a lung cancer, such as a small cell lung cancer. In some aspects, the tumor is a breast cancer. In some aspects, the tumor is a cervical cancer. In some aspects, the tumor is a colorectal cancer. In some aspects, the method further comprises administering to the subject an immunotherapy. In some aspects, the immunotherapy comprises an anti-PD-1 antibody or an antigen binding fragment thereof. In some aspects, following administration of the pharmaceutical composition, the subject does not experience necrosis in the liver, spleen, lungs, and/or kidneys. In some aspects, the administration of the pharmaceutical composition does not result in a cytokine storm, in particular, the administration of the pharmaceutical composition has no significant effect on the in vivo release of cytokines IL-1β, IL-2, IL-4, IL-10, IL-5, CXCL/KC and IL-12P/p70. In some aspects, the administration of the pharmaceutical composition does not increase liver function indicators glutamate transaminase (ALT) and glutathione transaminase (AST). In some aspects, the administration of the pharmaceutical composition does not increase leukocyte release, lymphocyte release, and/or monocyte release.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWING

FIG. 1A-FIG. 1D present a schematic representation of an IL-15 agnostic complex (FIG. 1A) and graphical representations of Bio-Layer Interferometry (BLI) results of the binding of FL115, ALT803 and FL115-WT, respectively, to IL-15R β (FIG. 1B-FIG. 1D) in accordance with an embodiment of the application, e.g., Example 2. FIG. 1A presents a schematic representation of IL-15 agonistic complex. FIG. 1B presents a graphical representation of FL115 binding to IL-15R β. FIG. 1C presents a graphical representation of ALT803 binding to IL-15R β. FIG. 1D presents a graphical representation of FL115-WT binding to IL-15R β. With reference to FIG. 1B-FIG. 1D, kon denotes the binding rate constant, koff denotes the dissociation rate constant, and KD represents kon/koff, which indicates the equilibrium constant of fusion protein binding to antigen that reflects the affinity of the fusion protein.

FIG. 2A-FIG. 2B present graphical representations of the biological activity of FL115 as detected by a CTLL2 cell proliferation assay (FIG. 2A) and a M-07e cell proliferation assay (FIG. 2B) in accordance with an embodiment of the application, e.g., Example 3. FIG. 2A presents a graphical representation of the proliferative activity of CTLL2 cells with the OD450 being measured as a function of the concentration of either FL115-V1 (squares), FL115-V2 (circles), or sFc (inverted triangles). FIG. 2B presents a graphical representation of the proliferative activity of M-07e cells with the luminescence being measured as a function of concentration of FL115.

FIG. 3A-FIG. 3E present graphical representations of the immunostimulatory effects of FL115 on human immune cells in accordance with an embodiment of the application, e.g., Example 4. FIG. 3A presents a graphical representation of a lymphocyte proliferation assay with the percentage proliferative response plotted against the concentration of ALT803 (squares) or FL115 (circles). FIG. 3B presents a graphical representation of an immune cell proliferation assay with the absolute cell number by cell type being presented for each of the medium (solid black), ALT803 (solid white), or FL115 (cross-hatch pattern). FIG. 3C presents a graphical representation of a cell surface marker activation assay with the CD69 MFI plotted against the concentration of ALT803 CD8+ T cells (squares), FL115 CD8+ T cells (hexagons), ALT803 NK cells (diamonds), FL115 NK cells (stars), FL115 CD4+ T cells (inverted triangles), or ALT803 CD4+ T cells (circles). FIG. 3D presents a graphical representation of a granzyme B expression assay with the MFI being plotted against the concentration of NK cells (circles) or CD8+ T cells (squares). FIG. 3E presents a graphical representation of a perforin expression assay with the MFI being plotted against the concentration of NK cells (circles) or CD8+ T cells (squares).

FIG. 4 presents a graphical representation of FL115 binding to FcRn as measured by surface plasmon resonance (SPR) in accordance with an embodiment of the application, e.g., Example 5.

FIG. 5A-FIG. 5E present graphical representations of the anti-tumor effect of FL115 in MC38 subcutaneous graft tumor model in accordance with an embodiment of the application, e.g., Example 6. FIG. 5A presents data obtained for the change in tumor volume over time for FL115 (circles); ALT803 (squares), and PBS (triangles). FIG. 5B presents data obtained for the change in tumor volume over time for FL115. FIG. 5C presents data obtained for the change in tumor volume over time for ALT803. FIG. 5D presents data obtained for the change in tumor volume over time for PBS. FIG. 5E presents data obtained for the change in weight over time for FL115 (circles); ALT803 (squares), and PBS (triangles).

FIG. 6A-FIG. 6D present graphical representations of assays measuring the killing function of NK cell and CD8+ T cells in tumor tissue of a MC38 mouse colon cancer cell subcutaneous transplantation tumor model in accordance with an embodiment of the application, e.g., Example 7. FIG. 6A presents results measuring GZMB+ CD8+ in CD3+ cells for PBS, ALT803, or FL115. FIG. 6B presents results measuring perforin+ CD8+ in CD3+ cells for PBS, ALT803, or FL115. FIG. 6C presents results measuring GZMB+ in NK cells for PBS, ALT803, or FL115. FIG. 6D presents results measuring perforin+ in NK cells for PBS, ALT803, or FL115.

FIG. 7 presents a graphical representation of a survival curve of mice in a CT26 mouse colon cancer metastatic tumor model who have been administered either PBS, ALT803, or FL115 in accordance with an embodiment of the application, e.g., Example 8. Note that the lines representing FL115 and ALT803 overlap in the graphical representation of FIG. 7.

FIG. 8A-FIG. 8D present graphical representations of the anti-tumor effect of FL115 in combination with anti-PD-1 antibody in mice with an embodiment of the application, e.g., Example 9. FIG. 8A presents results related to tumor volume over time in CMT167 mice treated with either aPBS (circles), FL115 (squares), PD1 (upright triangles), or FL115+PD-1 (inverted triangles). FIG. 8B presents results related to mouse weight over time in CMT167 mice treated with either PBS (circles), FL115 (squares), PD1 (upright triangles), or FL115+PD-1 (inverted triangles). FIG. 8C presents results related to tumor volume over time in LLC mice treated with either PBS (circles), FL115 (squares), PD1 (upright triangles), or FL115+PD-1 (inverted triangles). FIG. 8D presents results related to mouse weight over time in LLC mice treated with either PBS (circles), FL115 (squares), PD1 (upright triangles), or FL115+PD-1 (inverted triangles).

FIG. 9A-FIG. 9B present graphical representations of BLI validation of FL115 affinity for CD16a 158V and CD16a 158F in accordance with an embodiment of the application, e.g., Example 10. FIG. 9A presents a graphical representation of BLI validation of FL115 affinity for CD16a 158F. FIG. 9B presents a graphical representation of BLI validation of FL115 affinity CD16a 158V.

FIG. 10A-FIG. 10D present graphical representations of the effect of FL115 on liver and kidney function in a safety evaluation of mice in accordance with an embodiment of the application, e.g., Example 11. FIG. 10A presents a graphical representation of creatinine (ECRE) levels in mice administered a given dose of PBS, FL115, or ALT803. FIG. 10B presents a graphical representation of urea nitrogen (UN) levels in mice administered a given dose of PBS, FL115, or ALT803. FIG. 10C presents a graphical representation of glutathione transaminase (AST) levels in mice administered a given dose of PBS, FL115, or ALT803. FIG. 10D presents a graphical representation of glutamate transaminase (ALT) levels in mice administered a given dose of PBS, FL115, or ALT803.

FIG. 11A-FIG. 11D present graphical representations of the hematological effects of FL115 in mice during a safety evaluation in accordance with an embodiment of the application, e.g., Example 11. FIG. 11A presents the WBC count in mice administered a given dosage of PBS, FL115, or ALT803. FIG. 11B presents the lymphocyte count in mice administered a given dosage of PBS, FL115, or ALT803. FIG. 11C presents the monocyte count in mice administered a given dosage of PBS, FL115, or ALT803. FIG. 11D presents the neutrophil count in mice administered a given dosage of PBS, FL115, or ALT803.

FIG. 12A-FIG. 12F present graphical representations of the visceral weight of various organs in mice during safety evaluation of FL115 in accordance with an embodiment of the application, e.g., Example 11. FIG. 12A presents the lung weight of mice administered either PBS, FL115, or ALT803. FIG. 12B presents the lymph node weight of mice administered either PBS, FL115, or ALT803. FIG. 12C presents the spleen weight of mice administered either PBS, FL115, or ALT803. FIG. 12D presents the liver weight of mice administered either PBS, FL115, or ALT803. FIG. 12E presents the kidney weight of mice administered either PBS, FL115, or ALT803. FIG. 12F presents the heart weight of mice administered either PBS, FL115, or ALT803.

FIG. 13 presents an image of H&E results in the 20 mg/kg ALT 803 group in accordance with an embodiment of the application, e.g., Example 11.

FIG. 14 presents an image of the results of visceral histochemistry in mice from various different dosing groups in accordance with an embodiment of the application, e.g., Example 11.

FIG. 15A-FIG. 15J present graphical representations of FL115 stimulation of IL-6 (FIG. 15A), IFN-γ (FIG. 15B), TNF-α (FIG. 15C), IL-1β (FIG. 15D), IL-2 (FIG. 15E), IL-4 (FIG. 15F), IL-10 (FIG. 15G), IL-5 (FIG. 15H), CXCL/KC (FIG. 15I), and IL-12P/p70 (FIG. 15J) in mice in accordance with an embodiment of the application, e.g., Example 12. Open circles represent ALT803 at a dose of 20 mg/kg; diamonds represent ALT803 at a dose of 2 mg/kg; inverted triangles represent ALT803 at a dose of 0.2 mg/kg; upright triangles represent FL115 at a dose of 20 mg/kg; squares represent FL115 at a dose of 2 mg/kg; and circles represent FL115 at a dose of 0.2 mg/kg.

FIG. 16A-FIG. 16C present graphical representations of results of the anti-tumor effect of IL-15 in a subcutaneous mouse bladder cancer model in accordance with an embodiment of the application, e.g., Example 13. FIG. 16A presents a graphical representation of the change in tumor volume over time of mice administered either PBS (circles), FL115 at 0.2 mg/kg (squares), FL115 at 2 mg/kg (upright triangles), or FL115 at 20 mg/kg (inverted triangles). FIG. 16B presents a graphical representation of the tumor inhibition rate over time of mice administered either PBS (circles), FL115 at 0.2 mg/kg (squares), FL115 at 2 mg/kg (upright triangles), or FL115 at 20 mg/kg (inverted triangles). FIG. 16C presents a graphical representation of the change in body weight over time of mice administered either PBS (circles), FL115 at 0.2 mg/kg (squares), FL115 at 2 mg/kg (upright triangles), or FL115 at 20 mg/kg (inverted triangles)

FIG. 17A-FIG. 17C present data related to the anti-tumor effect of IL-15 in an orthotopic model of bladder cancer in mice in accordance with an embodiment of the application, e.g., Example 13. FIG. 17A presents the bladder weight of each mouse in four different groups of mice. FIG. 17B presents an image of the bladder excised from each mouse from the four different groups of mice. FIG. 17C presents a graphical representation of the body weight each mouse in from the four different groups of mice.

FIG. 18A-FIG. 18B present results on the measurement of the half-life of FL115 and ALT 803 in mice, e.g., Example 14. FIG. 18 A shows the serum concentrations of FL115 at various time points after their administrations to mice. FIG. 18B shows the serum concentrations of ALT 803 at various time points after their administrations to mice.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

Unless otherwise stated, any numerical value, such as a % sequence identity or a % sequence identity range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein, the terms “antibody” and “antibodies” refer to molecules that contain an antigen binding site. Antibodies include, but are not limited to, monoclonal antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and TgA2) or subclass.

As used herein, the terms “disease” and “disorder” are used interchangeably to refer to a condition affected by IL-15 signal transduction. In particular, a “disease” and “disorder” can be a pathological condition, and more particularly a disease affected by IL-15 signal transduction.

The term “agonist” refers to a compound that binds to a receptor and elicits an agonistic intracellular response. Agonists mimic the effects of endogenous ligands, hormones, and produce physiological responses similar to those produced, for example, by endogenous ligands.

As used herein, “fragment crystallizable region” or “Fc” refers to a polypeptide containing an antibody heavy chain constant region excluding the first domain of the constant region CH1. Therefore, Fc can include the last two domains of the heavy chain constant region (CH2 and CH3) of an IgA, IgD or IgG antibody, the last three domains of the heavy chain constant region (CH2, CH3 and CH4) of an IgE and IgM antibody. The Fc can also include the flexible hinge region connected to the N-terminus of the CH2 domain. For IgA and IgM, Fc can include the J chain. For IgG, Fc contains immunoglobulin domains Cγ2 and Cγ3 and the hinge between Cγ1 and Cγ2. Fc plays multiple roles in dimerization for the formation of Y-shaped structure of Ig and maintenance of the structure, and Fc-mediated effector functions and extension of serum half-life. The binding of Fe in IgG to its receptor Fc-gamma receptors (FcγRs) triggers antibody-dependent cell-mediated cytotoxicity (ADCC) and antibody-dependent cell-mediated phagocytosis to kill and clear target cells (e.g., tumor cells). The binding of Fe to the serum complement molecule (C1q) can initiate the assembly of membrane attack complex formed by complement cascade proteins to destroy target cells, which is termed complement-dependent cytotoxicity (CDC). Besides mediation of effector functions, Fc can also bind to neonatal Fc receptor (FcRn) in a pH-dependent manner, which can result in the extension of the serum half-life of IgG. In addition, binding of Fe to immune-related molecules such as Fc receptors can regulate immune response in vivo. Fc can refer to this region in isolation, or in the context of an antibody, antibody fragment, or Fc fusion. The Fc can be part of an antibody, an Fc fusion, or a protein or protein domain comprising Fc. The naturally occurring Fc forms a homodimer. Particularly preferred are Fc variants, which are non-naturally occurring Fc variants obtained using synthetic biology.

As used herein, “Fc monomer” or “monomeric Fc” refers to a Fc variant that no longer forms a homodimer under physiological conditions. Examples of Fc monomer include, but are not limited to, Fc monomers described in Wang et al., 2017, Front. Immunol., vol. 8, article 1545, WO2022088484A1, and US20210206847, the content of each of which is hereby incorporated by reference in its entirety.

In some aspects, the Fc monomer of the present disclosure comprises an Fc polypeptide whose molecular weight is only half of the wild-type Fe dimer, and retains the FcRn binding performance and Protein A/G binding performance of the antibody Fc region, and can achieve high-efficiency expression in host cells, such as prokaryotic host cells, and can significantly decrease non-specific binding as compared to previously developed monomeric IgG1 Fc mutants.

As used herein, the term “IL-15 functional fragment” refers to a portion of IL-15 that can serve to elicit or induce any degree of biological activity associated with IL-15. Such functional fragments include any mutated IL-15 of any length and/or any truncated version of IL-15 that retains a biological activity of IL-15. In some aspects, an IL-15 functional fragment comprises or consists of the sequence of SEQ ID NO: 3. In some aspects, an IL-15 functional fragment comprises or consists of the sequence of SEQ ID NO: 4. In some aspects, an IL-15 functional fragment comprises the sequence of any of the IL-15 sequences described throughout the present application.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

Herein, “autoimmune disease” refers to a disease in which the immune system produces an immune response (for example, a B cell or T cell response) against a part of an antigen of a normal host (i.e., an autoantigen), and then causes damage to the tissue. Autoantigens may be derived from host cells, or may be derived from symbiotic organisms, such as microorganisms that usually colonize mucosal surfaces (referred to as symbiotic organisms). Autoimmune diseases that affect mammals include but are not limited to rheumatoid arthritis, juvenile oligoarthritis, collagen-induced arthritis, adjuvant-induced arthritis, Sjogren's syndrome, multiple sclerosis, experimental Autoimmune encephalomyelitis, inflammatory bowel disease (e.g., Crohn's disease, ulcerative colitis), autoimmune gastric atrophy, pemphigus vulgaris, psoriasis, vitiligo, type 1 diabetes, non-obese diabetes, Myasthenia gravis, Graves' disease, Hashimoto's thyroiditis, sclerosing cholangitis, sclerosing salivitis, systemic lupus erythematosus, autoimmune thrombocytopenic purpura, Goodpasture syndrome, Addison's disease, systemic sclerosis, Polymyositis, dermatomyositis, autoimmune hemolytic anemia, pernicious anemia, etc.

“Virus” of the present disclosure include but is not limited to, viruses of the following families: Retroviridae (e.g., Human Immunodeficiency Virus (HIV), Human T-cell Leukemia Virus (HTLV)); Picornaviridae (e.g., Poliomyelitis) Virus, hepatitis A virus, hepatitis C virus, enterovirus, human Coxsackie virus, rhinovirus, Echo virus, foot-and-mouth disease virus); cadheroviridae (such as the virus strain that causes gastroenteritis); tunica Virus family (e.g. equine encephalitis virus, rubella virus); flaviviridae (e.g. dengue virus, yellow fever virus, West Nile virus, St. Louis encephalitis virus, Japanese encephalitis virus and other encephalitis viruses); coronavirus Family (e.g. coronavirus, severe acute respiratory syndrome (SARS) virus); Rhabdoviridae (e.g., vesicular stomatitis virus, rabies virus); Paramyxoviridae (e.g., parainfluenza virus, mumps virus, measles) Viruses, Respiratory Syncytial Virus (RSV); Orthomyxoviridae (e.g., influenza virus); Bunyaviridae (e.g. Hantavirus, SinNombre virus, Rift Valley fever virus, bunya virus, phleboviruses and Nairo virus); Arenaviridae (e.g. hemorrhagic fever virus, Machupo virus, Junin virus); reoviridae (e.g. reovirus, orbiviurse and rotavirus); binnaviridae; hepatoviridae (e.g. type B Hepatitis virus); Parvoviridae (e.g. parvovirus); Papovaviridae (e.g. papillomavirus, polyoma virus, BK virus); Adenoviridae (e.g., most adenoviruses, such as adeno-associated viruses); herpes Virus family (for example, herpes simplex virus (HSV-1 and HSV-2), cytomegalovirus (CMV), Epstein-Barr virus (EBV), varicella-zoster virus (VZV), and other herpes viruses, including HSV-6); poxvirus family (e.g., variola virus, vaccinia virus, poxvirus); and iris virus family (e.g. African swine fever virus); virus family (e.g., Ebola virus, Marburg virus); calicivirus Family (e.g., Norwalk virus) and unclassified viruses (e.g., pathogen of spongiform encephalopathy, pathogen of delta hepatitis (considered a defective satellite of hepatitis B virus), and astrovirus).

The “bacteria” of the present disclosure include but are not limited to: Helicobacter pylori, Borelia burgdorferi, Legionella pneumophila, Mycobacterium (such as M. tuberculosis, M. avium, M. intracellulare, M. kansaii, M. gordonae), gold Staphylococcus aureus, Neisseria gonorrhoeae, Neisseria meningitidis, Listeria monocytogenes, Streptococcus pyogenes (group A Streptococcus), Streptococcus agalactiae (group B Streptococcus), Streptococcus (lucky grass) Group), Streptococcus faecalis, Streptococcus bovis, Streptococcus (anaerobic bacteria), Streptococcus pneumoniae, Pathogenic Campylobacter, Enterococcus, Haemophilus influenzae, Bacillus anthracis, Corynebacterium diphtheria, Corynebacterium, Swine fever Viruses, Clostridium perfringens, Clostridium tetani, Enterobacter aerogenes, Klebsiella pneumoniae, Pasteurella multocida, Bacteroides, Clostridium, Streptomyces malt, Treponema pneumoniae, Treponema pneumoniae Spirochetes, Leptospira or Actinomyces (Actinomyces israelli).

The “fungi” of the present disclosure include but are not limited to: Cryptococcus neoformans, Histoplasmacapsulatum, Coccidioides immitis, Blastomycesdermatitidis, Chlamydia trachomatis or Candida albicans Bacteria (Candida albicans).

The “parasite” of the present disclosure includes but is not limited to: Plasmodium falciparum or Toxoplasma gondii.

“Cancer” as used herein refers to solid tumors or blood-borne cancers. The solid tumor of the present disclosure includes a sarcoma or cancer, such as fibrosarcoma, myxosarcoma, liposarcoma, chondrosarcoma, osteosarcoma, or another sarcoma, synovial tumor, mesothelioma, Ewing's tumor, leiomyosarcoma, Rhabdomyosarcoma, colon cancer, lymphoid malignancies, pancreatic cancer, breast cancer, lung cancer, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, Papillary adenocarcinoma, medullary carcinoma, bronchial carcinoma, renal cell carcinoma, hepatocellular carcinoma, cholangiocarcinoma, choriocarcinoma, Wilms tumor, cervical cancer, testicular tumor, bladder cancer or central nervous system tumor (such as glioma), Astrocytoma, medulloblastoma, craniopharyngioma, ependymoma, pineal gland, hemangioblastoma, acoustic neuroma, oligodendroglioma, hemangioma, melanoma, neuroblastoma Cell tumor or retinoblastoma). The blood-borne cancer of the present disclosure include leukemia, such as acute leukemia (such as acute lymphoblastic leukemia, acute myeloid leukemia, acute myeloid leukemia and myeloblasts, promyelocytic cells, myelomononuclear cells, monocytes and Erythroleukemia); chronic leukemia (such as chronic granulocytic (granulocyte) leukemia, chronic granulocytic leukemia and chronic lymphocytic leukemia), polycythemia vera, lymphoma, Hodgkin's disease, non-Hodgkin's lymphoma (inert And advanced forms), multiple myeloma, Waldenstrom's macroglobulinemia, heavy chain disease, myelodysplastic syndrome, hairy cell leukemia or myelodysplasia.

As used herein, “subject” means any animal, preferably a mammal, most preferably a human, to whom will be or has been treated by a method according to an embodiment of the present disclosure. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human. A human subject can include a patient.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

As used herein, the terms “treat”, “treating” and “treatment” in the context of the administration of a therapy to a subject refer to the beneficial effects that a subject derives from a therapy, such as, but not limited to, the reduction or inhibition of the progression, spread and/or duration of a disease or disorder, the reduction or amelioration of the severity of a disease or disorder, amelioration of one or more symptoms of a disease or disorder, and/or the reduction in the duration of one or more symptom of a disease or disorder resulting from the administration of one or more therapies. In specific embodiments, such terms in the context of cancer include, but are not limited to, one, two, or three or more results following the administration of a therapy to a subject: (1) a reduction in the growth of a tumor or neoplasm; (2) a reduction in the formation of a tumor; (3) an eradication, removal, or control of primary, regional and/or metastatic cancer; (4) a reduction in metastatic spread; (5) a reduction in mortality; (6) an increase in survival rate; (7) an increase in length of survival; (8) an increase in the number of patients in remission; (9) a decrease in hospitalization rate; (10) a decrease in hospitalization lengths; and (11) the maintenance in the size of the tumor so that it does not increase by more than 10%, or by more than 8%, or by more than 6%, or by more than 4%; preferably the size of the tumor does not increase by more than 2%.

As used herein, the terms “prevent,” “preventing” and “prevention” in the context of the administration of a therapy to a subject refer to the inhibition of the onset or recurrence of a disease or disorder in a subject.

The phrases “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The same determination may be made for nucleotide sequences. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g., using the NCBI BLAST algorithm (Altschul S F, et al (1997), Nucleic Acids Res. 25:3389-3402).

As used herein, a “non-naturally occurring” nucleic acid or polypeptide refers to a nucleic acid or polypeptide that does not occur in nature. A “non-naturally occurring” nucleic acid or polypeptide can be synthesized, treated, fabricated, and/or otherwise manipulated in a laboratory and/or manufacturing setting. In some cases, a non-naturally occurring nucleic acid or polypeptide can comprise a naturally-occurring nucleic acid or polypeptide that is treated, processed, or manipulated to exhibit properties that were not present in the naturally-occurring nucleic acid or polypeptide, prior to treatment. As used herein, a “non-naturally occurring” nucleic acid or polypeptide can be a nucleic acid or polypeptide isolated or separated from the natural source in which it was discovered, and it lacks covalent bonds to sequences with which it was associated in the natural source. A “non-naturally occurring” nucleic acid or polypeptide can be made recombinantly or via other methods, such as chemical synthesis.

As used herein, the term “operably linked” refer to a linkage or a juxtaposition wherein the components so described are in a relationship permitting them to function in their intended manner. For example, a regulatory sequence operably linked to a nucleic acid sequence of interest is capable of directing the transcription of the nucleic acid sequence of interest, or a signal sequence operably linked to an amino acid sequence of interest is capable of secreting or translocating the amino acid sequence of interest over a membrane.

In an attempt to help the reader of the present disclosure, the description has been separated in various paragraphs or sections or is directed to various embodiments of the present disclosure. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. The present disclosure contemplates use of any of the applicable components in any combination having any sequence that can be used in ribonucleic acid molecules of the present disclosure, whether or not a particular combination is expressly described.

IL-15 and Agonistic IL-15 Complexes

Though tumor immune checkpoint inhibitors have proven a milestone event in tumor therapy, the overall efficiency of such treatments is not high, and the objective response rate of single drug treatment is mostly in the range of 20%-30%. Based on the understanding of the tumor microenvironment, many researchers believe that immune activation against NK cells is an important way to solve the problem of low efficiency of PD-1 monoclonal antibodies.

A human IL-15, e.g., a 12-14 kD cytokine, was discovered by Grabstein et al. in 1994. It can play a role in the body's normal immune response, such as promoting the proliferation of T cells, B cells and NK cells. It has been demonstrated that IL-15 could be a useful therapeutic alternative treatment or combination treatment. For instance, IL-15 has a more biased NK cell activation activity than IL-2. In addition, IL-2 stimulates the proliferation of Treg in addition to the proliferation of killer immune cells, and these Treg suppress the immune response, leading to a diminished antitumor effect. In contrast, the specific receptor for IL-15 is IL-15Rα (CD125), which does not activate Treg cells. Therefore, from the perspective of being an immune activator, IL-15 has significant advantages over IL-2 and is expected to become a new generation of anti-tumor immune activator for 70-80% of tumor patients who do not respond to PD-1 monoclonal antibody, relying on its unique NK cell activation ability, with great market potential. As used herein, the term “IL-15” encompasses a full-length IL-15 or an IL-15 functional fragment. An “IL-15” can be a wild-type IL-15 or a variant IL-15.

In spite of its potential, the wild-type IL-15 has an extremely short half-life in vivo and very limited efficacy. In addition, IL-15 has a trans delivery mechanism, and effective IL-15 therapeutics need to consider the formation of complexes between IL-15 and IL-15Rα, which poses great difficulties. In addition to the short half-life and poor pharmacokinetics as factors limiting its clinical application, increasing the dosage of IL-15 can also induce a cytokine storm, leading to strong toxic side effects, thereby greatly limiting its clinical dosage level. Therefore, improved IL-15 therapies are of high interest and of great value.

As such, the present disclosure generally relates to a protein complex, i.e., an agonistic IL-15 complex, comprising an IL15 and a monomeric IL-15 receptor αSu/Fc fusion protein, and uses thereof. The agonistic IL-15 complexes described herein have a long half-life, have demonstrated good safety profiles, and have demonstrated higher anti-tumor activity as compared to other IL-15 based treatments. For instance, as demonstrated in the examples below, the in vivo distribution profile of an agonistic IL-15 complex according to an embodiment of the application was flatter than that of a reference (i.e., ALT 803), and the Cₘₐₓ of an agonistic IL-15 complex according to an embodiment of the application was not as high as that of the reference, resulting in not promoting a large release of cytokines, such as IL-6. Instead of containing a monomeric IL-15 receptor αSu/Fc fusion protein together with an IL15 as in the agonistic IL-15 complexes described herein, the reference ALT 803 is a protein complex containing a dimeric IL-15 receptor αSu/Fc fusion protein together with an IL15 (see, e.g., Xu et al., Cancer Res., 2013 May 15; 73(10):3075-86).

In one general aspect, the application relates to a fusion protein comprising an interleukin-15 (IL-15) receptor α sushi domain fused to an Fc monomer, wherein the IL-15 receptor α sushi domain comprises the amino acid sequence of SEQ ID NO: 5 and the Fc monomer comprises the amino acid sequence of SEQ ID NO: 6. In some embodiments, the carboxyl-terminus of the IL-15 receptor α sushi domain is fused to the amino-terminus of the Fc monomer via a linker having the amino acid sequence of GGGGS (SEQ ID NO: 7), such as a linker consisting of the amino acid sequence of SEQ ID NO: 7 or SEQ ID NO: 8. In certain embodiments, the fusion protein comprises or consists of the amino acid sequence of SEQ ID NO: 1. In certain embodiments, the fusion protein comprises or consists of the amino acid sequence of SEQ ID NO: 2.

In another general aspect, the application relates to a protein complex comprising a fusion protein according to an embodiment of the application and an IL-15. In some embodiments, the IL-15 comprises a functional IL-15 fragment having or consisting of the amino acid sequence of SEQ ID NO: 3. In some other embodiments, the IL-15 comprises a functional IL-15 fragment having or consisting of the amino acid sequence of SEQ ID NO: 4.

In some embodiments, the present disclosure generally relates to an agonistic interleukin-15 (IL-15) complex, wherein the agonistic IL-15 complex agonist comprises an Fc monomer, an IL-15 receptor α sushi domain and an IL-15 functional fragment, wherein the Fc monomer is fused to the IL-15 receptor α sushi domain, wherein the Fc monomer-IL-15 receptor α sushi domain fusion comprises the sequence of SEQ ID NO: 1 or SEQ ID NO: 2, and further wherein the IL-15 functional fragment comprises the sequence of SEQ ID NO: 3 or SEQ ID NO: 4. In some aspects, the Fc monomer-IL-15 receptor α sushi domain fusion comprises the sequence of SEQ ID NO: 1, and the IL-15 functional fragment comprises the sequence of SEQ ID NO: 3. In some aspects, the Fc monomer-IL-15 receptor α sushi domain fusion comprises the sequence of SEQ ID NO: 1, and the IL-15 functional fragment comprises the sequence of SEQ ID NO: 4. In some aspects, the Fc monomer-IL-15 receptor α sushi domain fusion comprises the sequence of SEQ ID NO: 2, and the IL-15 functional fragment comprises the sequence of SEQ ID NO: 3. In some aspects, the Fc monomer-IL-15 receptor α sushi domain fusion comprises the sequence of SEQ ID NO: 2, and the IL-15 functional fragment comprises the sequence of SEQ ID NO: 4.

In some aspects, the C-terminus of the IL-15 receptor α-sushi domain is connected to the Fc monomer, and the IL-15 receptor α-sushi domain is capable of binding to the IL-15 functional fragment. In some aspects, the C-terminus of the IL-15 receptor α-sushi domain is connected to the Fc monomer by a covalent bond; preferably, the Fc monomer and the IL-15 receptor α sushi domain are connected by a connecting peptide, and more preferably, the connecting peptide is GGGGS or (GGGGS)3. The connecting peptide refers to a polypeptide chain containing flexible amino acid residues, and the flexible amino acid residues are Gly, Ser, Ala, or Thr. The polypeptide chain should have a suitable distance, which is suitable for connecting two molecules so that they have the correct configuration with respect to each other to maintain the desired activity. Suitable lengths for this purpose include at least one and no more than 30 amino acid residues. Preferably, the length of the linker is about 1-30 amino acids, and the preferred length of the linker is 1, 2, 3, 4, 5, 6, 7, 8, 9, 10, 11, 12, 13, 14, 15, 16, 17, 18, 19 and 20 amino acids. In addition, the properties shown by the amino acid residues selected for inclusion in the connecting peptide should not significantly affect the activity of the two linked molecules. Therefore, the connecting peptide generally does not have a charge that is inconsistent with the two connected molecules, or does not affect internal folding, or forms bonds or other interactions with amino acid residues in one or more monomers such that the monomer would severely hinder the binding of the receptor monomer domain. In some embodiments, the connecting peptides contain flexible amino acid residues. For example, the connecting peptide can include a glycine-serine polymer, such as (GS)n, (GSGGS)n, (GGGGS)n, (GGGS)n, where n is an integer of at least 1, a glycine-alanine polymer, an alanine-serine polymer, and other flexible connecting peptides known in the art such as the connecting sequence of the shaker potassium channel, etc.

In some aspects, the IL-15 receptor α sushi domain of the present disclosure comprises the extracellular domain of IL-15 receptor α starts from the cysteine residue (C1) encoded by the first exon 2 and ends at the fourth exon. The cysteine residue (C4), residues C1 and C4 encoded by sub-2 are all contained in the sushi domain. The amino acid sequence of the α sushi domain of IL-15 receptor formed by the substitution, deletion or addition of one or more amino acid residues and having corresponding activity is also included in the present disclosure.

In some aspects, the agonistic IL-15 complex binds to IL-15 receptor β (IL-15Rβ). In some aspects, the agonistic IL-15 complex binds to IL-15Rβ with a Kd of 9.00×10⁻⁹ M or less, 8.00×10⁻⁹ M or less, 7.00×10⁻⁹ M or less, 6.00×10⁻⁹ M or less, 5.00×10⁻⁹ M or less, 4.75×10⁻⁹ M or less, 4.50×10⁻⁹ M or less, or 4.47×10⁻⁹ M or less. In some aspects, the agonistic IL-15 complex binds to FcRn. In some aspects, the agonistic IL-15 complex is capable of binding to FcRn with an affinity of 9.00×10⁻⁶ M or less, 8.00×10⁻⁶ M or less, 7.00×10⁻⁶ M or less, 6.00×10⁻⁶ M or less, 5.00×10⁻⁶ M or less, 4.75×10⁻⁶ M or less, 4.50×10⁻⁶ M or less, 4.25×10⁻⁶ M or less, or 4.07×10⁻⁶ M or less. In some aspects, the agonistic IL-15 complex is capable of stimulating the proliferation of M-07e cells. In some aspects, the agonistic IL-15 complex is capable of inducing granzyme secretion from tumor-infiltrating killer NK cells and/or CD8+ T cells. Granzymes are a family of serine proteases that induce cell death mediated by a collective of cytotoxic lymphocytes (CLs) (e.g. cytotoxic T lymphocytes (CTLs), natural killer (NK) cells). In some aspects, the agonistic IL-15 complex is not capable of binding to CD16a 158V or CD16a 158F. In some aspects, the Fc monomer-IL-15 receptor α sushi domain fusion is capable of binding to the IL-15 functional fragment. In some aspects, the IL-15 functional fragment is an endogenously expressed IL-15 functional fragment. In some aspects, the IL-15 functional fragment is a mutant IL-15 fragment. In some aspects, an Fc monomer-IL-15 receptor α sushi domain fusion is capable of extending the half-life of IL-15 or an IL-15 functional fragment in vivo.

Nucleic Acid Molecules, Polynucleotides, and Host Cells

The present disclosure further generally relates to a nucleic acid molecule encoding the agonistic IL-15 complex as described herein, wherein the Fc monomer-IL-15 receptor α sushi domain fusion and the IL-15 functional fragment are encoded by polynucleotides. In some aspects, the Fc monomer-IL-15 receptor α sushi domain fusion and the IL-15 functional fragment are encoded by separate polynucleotides. In some aspects, the Fc monomer-IL-15 receptor α sushi domain fusion and the IL-15 functional fragment are encoded by the same polynucleotide. In some aspects, the Fc monomer-IL-15 receptor α sushi domain fusion and the IL-15 functional fragment are present in the same vector. In some aspects, the Fc monomer-IL-15 receptor α sushi domain fusion and the IL-15 functional fragment are present in separate vectors. In some aspects, the Fc monomer-IL-15 receptor α sushi domain fusion and the IL-15 functional fragment are each operably linked to different promoters. As used herein, a “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. A vector can be a DNA vector or an RNA vector. One of ordinary skill in the art can construct a vector of the application through standard recombinant techniques in view of the present disclosure. A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

Vectors of the application can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some embodiments of the application, a vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g., a linear covalently closed DNA, e.g., a linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of suitable DNA plasmids that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli pYES2 (Invitrogen, Thermo Fisher Scientific), which can be used for production and/or expression in Saccharomyces cerevisiae strains of yeast; MAXBAC® complete baculovirus expression system (Thermo Fisher Scientific), which can be used for production and/or expression in insect cells; pcDNA™ or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

A vector of the application, e.g., a DNA plasmid or a viral vector, can comprise any regulatory elements to establish conventional function(s) of the vector. Regulatory elements include, but are not limited to, a promoter, an enhancer, a polyadenylation signal, translation stop codon, a ribosome binding element, a transcription terminator, selection markers, origin of replication, etc. A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest from a start codon to a stop codon. As used herein, the term “operably linked” is to be taken in its broadest reasonable context, and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

A vector can comprise a promoter sequence, preferably within an expression cassette, to control expression. The term “promoter” is used in its conventional sense, and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous).

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metallothionein. A promoter can also be a tissue specific promoter, such as a muscle or skin specific promoter, natural or synthetic.

Furthermore, the present disclosure generally relates to a host cell comprising a nucleic acid molecule, plasmid, and/or vector as described herein. The host cell of the present disclosure can be any prokaryotic cell or eukaryotic cell, including but not limited to bacterial cells (e.g. Escherichia coli, Bacillus subtilis), insect cells (e.g. using baculovirus expression systems), yeast or mammalian cells (e.g. CHO or BHK cell line). Other suitable host cells are known to those skilled in the art.

Pharmaceutical Compositions

The present disclosure additionally generally relates to a pharmaceutical composition comprising a fusion protein as described herein, a nucleic acid molecule as described herein, a plasmid as described herein, and/or a host cell as described herein, and a pharmaceutically acceptable excipient, diluent, or carrier. In some aspects, the pharmaceutical composition comprises a protein complex as described herein. In some aspects, the pharmaceutical composition further comprises one or more anti PD-1 antibodies or antigen binding fragments thereof and/or anti PD-L1 antibodies or antigen binding fragments thereof.

Typically, the administration of pharmaceutical compositions and therapeutic combinations of the present disclosure will have a therapeutic aim to generate an immune response against a disease, such as a cancer. As used herein, “an effective amount” or “a therapeutically effective amount” refer to an amount of an agonistic IL-15 complex as described herein or a composition comprising said complex sufficient to induce a desired immune effect or immune response or therapeutic effect in a subject in need thereof. A therapeutically effective amount can be an amount sufficient to induce an immune response in a subject in need thereof. A therapeutically effective amount can be an amount sufficient to produce immunity in a subject in need thereof, e.g., provide a therapeutic effect against a disease such a cancer. A therapeutically effective amount can vary depending upon a variety of factors, such as the physical condition of the subject, age, weight, health, etc.; the particular application, e.g., providing protective immunity or therapeutic immunity; and the particular disease, e.g., viral infection, for which immunity is desired. A therapeutically effective amount can readily be determined by one of ordinary skill in the art in view of the present disclosure.

Compositions and therapeutic combinations of the present disclosure, such as agonistic IL-15 complex in combination with an anti-PD-1 and/or anti-PD-L antibody or fragment thereof, can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. Pharmaceutically acceptable carriers can include vehicles, such as lipid (nano)particles. The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal spray s/inhalant mixtures, the aqueous solution/suspension can comprise water, glycols, oils, emollients, stabilizers, wetting agents, preservatives, aromatics, flavors, and the like as suitable carriers and additives.

Compositions and therapeutic combinations of the application, that is, those comprising an agonistic IL-15 complex, can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions of the application can also be formulated for other routes of administration including transmucosal, ocular, rectal, long-acting implantation, sublingual administration, under the tongue, from oral mucosa bypassing the portal circulation, inhalation, or intranasal.

According to aspects of the present disclosure, compositions and therapeutic combinations for administration will typically comprise a buffered solution in a pharmaceutically acceptable carrier, e.g., an aqueous carrier such as buffered saline and the like, e.g., phosphate buffered saline (PBS). The compositions and therapeutic combinations can also contain pharmaceutically acceptable substances as required to approximate physiological conditions such as pH adjusting and buffering agents. For example, a composition or therapeutic combination of the application comprising plasmid DNA can contain phosphate buffered saline (PBS) as the pharmaceutically acceptable carrier.

Methods of Treatment

The present disclosure further generally relates to a method of treating a disease in a subject in need thereof, comprising administering to the subject an effective amount of a pharmaceutical composition as described herein. In some aspects, the method further comprises administering to the subject another therapeutically active agent. In some aspects, the other therapeutically active agent comprises a small molecule compound, a targeted chemotherapeutic agent, a radiation therapy agent, or an antibody or an antigen binding fragment thereof. In some aspects, the other therapeutically active agent comprises an antibody or an antigen binding fragment thereof that binds specifically to CD20, PD1, PDL1, Her2, EGFR or c-MET. In some aspects, the other therapeutically active agent comprises an anti-PD-1 antibody or an antigen binding fragment thereof.

Moreover, the present disclosure further generally relates to a method for treating a disease in a subject in need thereof comprising administering to the subject an effective amount of an agonistic IL-15 complex as described herein, a nucleic acid molecule as described herein, a plasmid as described herein, a host cell as described herein, or a pharmaceutical composition as described herein. In some aspects, the method further comprises administering one or more anti PD-1 antibodies or antigen binding fragments thereof and/or anti PD-L1 antibodies or antigen binding fragments thereof. In some aspects, the agonistic IL-15 complex and the one or more anti PD-1 antibodies or antigen binding fragments thereof and/or anti PD-L1 antibodies or antigen binding fragments thereof is simultaneous administration. In some aspects, the administration of the agonistic IL-15 complex and the one or more anti PD-1 antibodies or antigen binding fragments thereof and/or anti PD-L1 antibodies or antigen binding fragments thereof is sequential administration. In some aspects, the agonistic IL-15 complex is used in combination with a small molecule inhibitor or antibody analog. In some aspects, the small molecule inhibitor is a targeted chemotherapeutic agent or radiation therapy agent and said antibody analog is an anti-CD20, PD1, PDL1, Her2, EGFR or c-MET antibody.

In some aspects, the subject is a mammal, preferably a human. In some aspects, the disease is a tumor. In some aspects, the tumor is a solid tumor. In some aspects, the disease is cancer. In some aspects, the cancer is leukemia, lymphoma, multiple myeloma, malignant melanoma, breast cancer, lung cancer, liver cancer, pancreatic cancer, prostate cancer, colon cancer, or renal cell carcinoma. In some aspects, the cancer is colon cancer or lung cancer. In some aspects, the cancer of the present disclosure includes, but is not limited to, lymphoma, blastoma, sarcoma (including liposarcoma), neuroendocrine tumor, mesothelioma, schwannoma, meningioma, adenoma, melanoma, and non-leukemic leukemia or Lymphoid malignancy. More specific examples of the aforementioned cancers include squamous cell carcinoma (e.g., squamous cell carcinoma), lung cancer, small cell lung cancer, non-small cell lung cancer, lung adenocarcinoma, and lung squamous cell carcinoma, peritoneal cancer, hepatocellular carcinoma, and gastric cancer, gastrointestinal cancer, pancreatic cancer, malignant glioma, cervical cancer, ovarian cancer, liver cancer, bladder cancer, hepatocellular tumor, breast cancer, colon cancer, rectal cancer, colorectal cancer, endometrial or uterine cancer, salivary gland cancer, kidney cancer, prostate cancer, vulvar cancer, thyroid cancer, liver cancer, anal cancer, penile cancer, testicular cancer, esophageal cancer, bile duct tumors, head cancer, neck cancer, bone marrow stromal tumor, osteoclastoma, multiple myeloma, Osteolytic bone cancers, central nervous system tumors, brain tumors (glioma, neuroblastoma, astrocytoma, medulloblastoma, ependymoma, and retinal neuroblastoma), Nasopharyngeal carcinoma, basal cell carcinoma, cholangiocarcinoma, Kaposi's sarcoma, primary liver cancer or endometrial cancer, and tumors of the vascular system (angiosarcoma and hemangiopericytomas).

In some aspects, the method comprises treating a pathogen infection. The pathogens of the present disclosure include but are not limited to bacteria, fungi, viruses, and parasites. In some aspects, the pathogen infection is a bacterial infection and/or a viral infection. In some aspects, the viral infection comprises infection with an immunodeficiency virus, a smallpox virus, a hepatitis B virus, or any combination of the foregoing. In some aspects, the viral infection is an HIV infection.

In some aspects, the disease is an autoimmune disease. In some aspects, the disease is an inflammatory disease. In some aspects, the disease is a neurodegenerative disease. In some aspects, following administration of the agonistic IL-15 complex, the subject experiences no inflammation or mild inflammation of the liver, spleen, lungs, and/or kidneys as compared to a patient administered ALT803 and/or as compared to a negative control. In some aspects, administration of the agonistic IL-15 complex does not result in a cytokine storm. In some aspects, administration of the agonistic IL-15 complex has no significant effect on the in vivo release of cytokines IL-1β, IL-2, IL-4, IL-10, IL-5, CXCL/KC and IL-12P/p70 as compared to a patient administered ALT803 and/or as compared to a negative control. In some aspects, administration of the agonistic IL-15 complex does not increase liver function indicators glutamate transaminase (ALT) and glutathione transaminase (AST) as compared to a subject administered a negative control and/or as compared to a subject administered ALT803. In some aspects, administration of the agonistic IL-15 complex does not increase leukocyte release, increase lymphocyte release, and/or increase monocyte release as compared to a subject administered a negative control and/or a subject administered ALT803.

In some aspects, the subject is administered 0.1 mg/kg, 0.20 mg/kg, 0.30 mg/kg, 0.40 mg/kg, 0.50 mg/kg, 0.60 mg/kg, 0.70 mg/kg, 0.80 mg/kg, 0.90 mg/kg, 1.00 mg/kg, 1.50 mg/kg, 2.00 mg/kg, 2.50 mg/kg, 3.00 mg/kg, 3.50 mg/kg, 4.00 mg/kg, 4.50 mg/kg, 5.00 mg/kg, 6.00 mg/kg, 7.00 mg/kg, 8.00 mg/kg, 9.00 mg/kg, 10.0 mg/kg, 12.5 mg/kg, 15.0 mg/kg, 17.5 mg/kg, 20.0 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg of the agonistic IL-15 complex. In some aspects, the agonistic IL-15 complex is administered subcutaneously, intramuscularly, or parenterally.

In some aspects, the agonistic IL-15 complex is administered to the subject as a part of a cellular immunotherapy. In some aspects, the cellular immunotherapy is CAR-T, TCR-T, DC, CIK, DC-CIK, ECIK, NK, CAS-T or BiAb-T tumor cellular immunotherapy. In some aspects, administration of the agonistic IL-15 complex induces function of NK cell and CD8+ T cells in tumor tissue.

In some aspects, an agonistic IL-15 complex as described herein can be used to treat conditions specifically including but not limited to: congestive heart failure (CHF), vasculitis, rosacea, acne, eczema, myocarditis and other myocardial disorders, systemic lupus erythematosus, diabetes, Spondylosis, synovial fibroblast hyperplasia, bone loss, paget's disease, apraxia osteopenia, malnutrition, periodontal disease, familial splenic anemia, Langham's cell histiocytosis, Spinal cord injury, acute septic arthritis, osteomalacia, hypercortisolism, single bone fibrous bone dysplasia, multiple bone fibrous dysplasia, periodontal reconstruction and fractures, sarcoidosis, bone metastases/bone Pain treatment and body fluid malignant hypercalcemia, ankylosing spondylitis and other spondyloarthropathy, transplant rejection, viral infection, hematoma, Hodgkin's lymphoma, non-Hodgkin's lymphoma (Burkitt's lymphoma, small lymphocytes) Lymphoma/chronic lymphocytic leukemia, granuloma fungoides, mantle cell lymphoma, follicular lymphoma, diffuse giant B-cell lymphoma, marginal zone lymphoma, hairy cell leukemia, and lymphoplasmacytic leukemia), Lymphocyte precursor cell tumor, B-cell acute lymphoblastic non-leukemic leukemia/lymphoma, T-cell acute lymphoblastic non-leukemic leukemia/lymphoma, thymoma, mature T and NK cell tumors, peripheral T Cell non-leukemic leukemia, mature T-cell non-leukemic leukemia/T-cell lymphoma, large granular lymphocytic leukemia, Langham's cell histiocytosis, acute myelogenous leukemia myeloma, mature acute Myelogenous leukemia (AML), differentiated acute myeloid leukemia, acute promyelocytic leukemia, acute myelomonocytic leukemia, acute monocytic leukemia, myelodysplastic syndrome, chronic myelodysplastic, chronic myeloid cells Leukemia, osteoporosis, hepatitis, HIV, AIDS, spondylarthritis, rheumatoid arthritis, inflammatory bowel disease (IBD), sepsis and septic shock, Crohn's disease, psoriasis, scleroderma, Graft versus host disease (GVHD), allogenic islet graft rejection (allogenic islet graft rejection), hematological malignancies such as multiple myeloma (MM), myelodysplastic syndrome (MDS) and acute myelogenous leukemia (AML), tumor-related Inflammation, peripheral nerve injury or demyelinating disease.

Furthermore, the present disclosure generally relates to a method of treating a tumor, comprising administering to a subject in need thereof an effective amount of the pharmaceutical composition as described herein. In some aspects, the tumor is a solid tumor, such as a carcinoma, a sarcoma, or a melanoma. In some aspects, the solid tumor is selected from the group consisting of a bladder cancer, a lung cancer, a breast cancer, a colorectal cancer, a brain tumor, a prostate cancer, a melanomas, a merkel cell carcinoma, a head and neck cancer, a small bowel cancer, a squamous cell carcinoma, metastatic solid tumor or a cervical cancer. In some aspects, the tumor is a bladder cancer. In some aspects, the tumor is a lung cancer, such as a small cell lung cancer. In some aspects, the tumor is a breast cancer. In some aspects, the tumor is a cervical cancer. In some aspects, the tumor is a colorectal cancer. In some aspects, the method further comprises administering to the subject an immunotherapy. In some aspects, the immunotherapy comprises an anti-PD-1 antibody or an antigen binding fragment thereof. In some aspects, following administration of the pharmaceutical composition, the subject does not experience necrosis in the liver, spleen, lungs, and/or kidneys. In some aspects, the administration of the pharmaceutical composition does not result in a cytokine storm, in particular, the administration of the pharmaceutical composition has no significant effect on the in vivo release of cytokines IL-1β, IL-2, IL-4, IL-10, IL-5, CXCL/KC and IL-12P/p70. In some aspects, the administration of the pharmaceutical composition does not increase liver function indicators glutamate transaminase (ALT) and glutathione transaminase (AST). In some aspects, the administration of the pharmaceutical composition does not increase leukocyte release, lymphocyte release, and/or monocyte release.

In addition to treatment of human disorders, agonistic IL-15 complexes as described herein will have significant use for veterinary applications, e.g., treatment of disorders of livestock such as cattle, sheep, etc. and pets such as dog and cats.

Methods of Production

The present disclosure further generally relates to a method of producing a fusion protein as discussed herein, the method comprising culturing a host cell as described herein under conditions sufficient to express the fusion protein, and isolating the fusion protein.

Furthermore, the present disclosure generally relates to a method of producing a protein complex as described herein, the method comprising culturing one or more host cells as described herein under conditions sufficient to express the fusion protein and the IL15, isolating the fusion protein and IL15, and optionally assembling the protein complex by combining the isolated fusion protein and IL15 in vitro.

Moreover, the present disclosure generally relates to a method of producing the agonistic IL-15 complex, comprising transfecting a cell with a nucleic acid molecule comprising the Fc monomer-IL-15 receptor α sushi domain fusion and the IL-15 functional fragment; b. culturing the host cell under conditions sufficient to express the Fc monomer-IL-15 receptor α sushi domain fusion and the IL-15 functional fragment; c. expressing the Fc monomer-IL-15 receptor a sushi domain fusion and the IL-15 functional fragment; d. in vitro assembly of the agonistic IL-15 complex; and e. purifying agonistic IL-15 complex. In some aspects, the Fc monomer-IL-15 receptor α sushi domain fusion and the IL-15 functional fragment are encoded by separate nucleic acid molecules. In some aspects, the Fc monomer-IL-15 receptor α sushi domain fusion and the IL-15 functional fragment are encoded by the same nucleic acid molecule.

Detection Kit

The present disclosure also generally relates to a detection kit comprising of an agonistic IL-15 complex as described herein, a nucleic acid molecule as described herein, a plasmid as described herein, a host cell as described herein, or a pharmaceutical composition as described herein, wherein the detection kit is optionally used to detect pathogens and/or tumor cells.

### Embodiments

Embodiment 1. A fusion protein comprising an interleukin-15 (IL-15) receptor α sushi domain fused to an Fc monomer, wherein the IL-15 receptor α sushi domain comprises the amino acid sequence of SEQ ID NO: 5 and the Fc monomer comprises the amino acid sequence of SEQ ID NO: 6.

Embodiment 2a. The fusion protein of embodiment 1, wherein the carboxyl-terminus of the IL-15 receptor α sushi domain is fused to the amino-terminus of the Fc monomer via a linker, such as a glycine-serine polymer, such as (GS)n, (GSGGS)n, (GGGGS)n, (GGGS)n, where n is an integer of at least 1, a glycine-alanine polymer, or an alanine-serine polymer.

Embodiment 2. The fusion protein of embodiment 1, wherein the carboxyl-terminus of the IL-15 receptor α sushi domain is fused to the amino-terminus of the Fc monomer via a linker having the amino acid sequence of GGGGS (SEQ ID NO: 7).

Embodiment 3. The fusion protein of embodiment 1, wherein the linker consists of the amino acid sequence of SEQ ID NO: 7 or SEQ ID NO: 8.

Embodiment 4. The fusion protein of embodiment 3, comprising the amino acid sequence of SEQ ID NO: 1.

Embodiment 5. The fusion protein of embodiment 3, comprising the amino acid sequence of SEQ ID NO: 2.

Embodiment 6. A fusion protein consisting of the amino acid sequence of SEQ ID NO: 1.

Embodiment 7. A fusion protein consisting of the amino acid sequence of SEQ TED NO: 2.

Embodiment 8. A protein complex comprising the fusion protein of any one of embodiments 1-7 and an IL-15.

Embodiment 9a. The protein complex of embodiment 8, wherein the IL-15 comprises a full-length IL-15 or an IL-15 functional fragment.

Embodiment 9b. The protein complex of embodiment 8 or 9a, wherein the IL-15 comprises a wild-type IL-15, a variant IL-15, or a functional fragment thereof.

Embodiment 9. The protein complex of embodiment 8, wherein the IL-15 comprises the amino acid sequence of SEQ ID NO: 3

Embodiment 10. The protein complex of embodiment 8, wherein the IL-15 consists of the amino acid sequence of SEQ ID NO: 3.

Embodiment 11. The protein complex of embodiment 8, wherein the IL-15 comprises the amino acid sequence of SEQ ID NO: 4.

Embodiment 12. The protein complex of embodiment 8, wherein the IL-15 consists of the amino acid sequence of SEQ ID NO: 4.

Embodiment 13. A nucleic acid molecule, such as a vector, encoding the fusion protein of any one of embodiments 1-7.

Embodiment 13a. A vector, such as a plasmid, a DNA vector, an RNA vector or a viral vector, encoding the fusion protein of any one of embodiments 1-7.

Embodiment 14. A host cell comprising the nucleic acid molecule of embodiment 13.

Embodiment 14a. A host cell comprising the vector of embodiment 13a.

Embodiment 15. A method of producing the fusion protein of any one of embodiments 1-7, comprising culturing the host cell of embodiment 14 under conditions sufficient to express the fusion protein, and isolating the fusion protein.

Embodiment 15a. A method of producing the fusion protein of any one of embodiments 1-7, comprising culturing the host cell of embodiment 14a under conditions sufficient to express the fusion protein, and isolating the fusion protein.

Embodiment 16. One or more nucleic acid molecules, such as one or more vectors, encoding the fusion protein and the IL-15 of the protein complex of any one of embodiments 8-12.

Embodiment 16a. One or more vectors, such as one or more plasmids, DNA vectors, RNA vectors or viral vectors, encoding the fusion protein and the IL-15 of the protein complex of any one of embodiments 8-12.

Embodiment 17. One or more host cells comprising the one or more nucleic acid molecules of embodiment 16.

Embodiment 17a. One or more host cells comprising the one or more vectors of embodiment 16a.

Embodiment 18. A method of producing the protein complex of any one of embodiments 8-12, comprising culturing the one or more host cells of embodiment 17 under conditions sufficient to express the fusion protein and the IL15, isolating the fusion protein and IL15, and optionally assembling the protein complex by combining the isolated fusion protein and IL15 in vitro.

Embodiment 18a. A method of producing the protein complex of any one of embodiments 8-12, comprising culturing the one or more host cells of embodiment 17a under conditions sufficient to express the fusion protein and the IL15, isolating the fusion protein and IL15, and optionally assembling the protein complex by combining the isolated fusion protein and IL15 in vitro.

Embodiment 19. A pharmaceutical composition comprising the fusion protein of any one of embodiments 1-7, the protein complex of any one of embodiments 8-12, the nucleic acid molecule of embodiment 13, the one or more nucleic acid molecules of embodiment 16, the host cell of embodiment 14, or the one or more host cells of embodiment 17, and a pharmaceutically acceptable excipient, diluent, or carrier.

Embodiment 20. The pharmaceutical composition of embodiment 19, comprising the protein complex of any one of embodiments 8-12.

Embodiment 21. A method of treating a disease in a subject in need thereof, comprising administering to the subject an effective amount of the pharmaceutical composition of embodiment 19 or embodiment 20.

Embodiment 22. The method of embodiment 21, further comprising administering to the subject another therapeutically active agent.

Embodiment 23. The method of embodiment 22, wherein the other therapeutically active agent comprises a small molecule compound, a targeted chemotherapeutic agent, a radiation therapy agent, or an antibody or an antigen binding fragment thereof.

Embodiment 24. The method of embodiment 23, wherein the other therapeutically active agent comprises an antibody or an antigen binding fragment thereof that binds specifically to CD20, PD1, PDL1, Her2, EGFR or c-MET.

Embodiment 25. The method of embodiment 24, wherein the other therapeutically active agent comprises an anti-PD-1 antibody or an antigen binding fragment thereof.

Embodiment 26. The method of any one of embodiments 21-25, wherein the disease is a tumor.

Embodiment 27. The method of embodiment 26, wherein the tumor is a solid tumor.

Embodiment 28. The method of any one of embodiments 21-25, wherein the disease is a pathogen infection, such as a bacterial infection and/or a viral infection.

Embodiment 29. The method of any one of embodiments 21-25, wherein the disease is an autoimmune disease.

Embodiment 30. The method of any one of embodiments 21-25, wherein the disease is an inflammatory disease.

Embodiment 31. The method of any one of embodiments 21-25, wherein the disease is a neurodegenerative disease.

Embodiment 32. A method of treating a tumor, comprising administering to a subject in need thereof an effective amount of the pharmaceutical composition of embodiment 20.

Embodiment 33. The method of embodiment 32, wherein the tumor is a solid tumor, such as a carcinoma, a sarcoma, or a melanoma.

Embodiment 34. The method of embodiment 33, wherein the solid tumor is selected from the group consisting of a bladder cancer, a lung cancer, a breast cancer, a colorectal cancer, a brain tumor, a prostate cancer, a melanomas, a merkel cell carcinoma, a head and neck cancer, a small bowel cancer, a squamous cell carcinoma, metastatic solid tumor or a cervical cancer.

Embodiment 35. The method of embodiment 34, wherein the tumor is a bladder cancer.

Embodiment 36. The method of embodiment 34, wherein the tumor is a lung cancer, such as a small cell lung cancer.

Embodiment 37. The method of embodiment 34, wherein the tumor is a breast cancer.

Embodiment 38. The method of embodiment 34, wherein the tumor is a cervical cancer.

Embodiment 39. The method of embodiment 34, wherein the tumor is a colorectal cancer.

Embodiment 40. The method of any one of embodiments 32-39, further comprising administering to the subject an immunotherapy.

Embodiment 41. The method of embodiment 40, wherein the immunotherapy comprises an anti-PD-1 antibody or an antigen binding fragment thereof.

Embodiment 42. The method of any one of embodiments 32-41, wherein, following administration of the pharmaceutical composition, the subject does not experience necrosis in the liver, spleen, lungs, and/or kidneys.

Embodiment 43. The method of any one of embodiments 32-41, wherein the administration of the pharmaceutical composition does not result in a cytokine storm, in particular, the administration of the pharmaceutical composition has no significant effect on the in vivo release of cytokines IL-10, IL-2, IL-4, IL-10, IL-5, CXCL/KC and IL-12P/p70.

Embodiment 44. The method of any one of embodiments 32-43, wherein the administration of the pharmaceutical composition does not increase liver function indicators glutamate transaminase (ALT) and glutathione transaminase (AST).

Embodiment 45. The method of any one of embodiments 32-44, wherein the administration of the pharmaceutical composition does not increase leukocyte release, lymphocyte release, and/or monocyte release.

SEQUENCE LISTING

| SEQ ID | | |
|---|---|---|
| NO | Description | Sequence |
| 1 | IL 15Rα- | ITCPPPMSVEHADIWVKSYSLYSRERYICNSGFKRKAGTSSLTECVLNK |
| | GGGGS-sFc | ATNVAHWTTPSLKCIRGGGGSAPELLGGPSVFLFPPKPKDTLMISRTPE |
| | | VTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLP |
| | | PSRDELTKNQVSLRCHVKGFYPSDIAVEWESNGQPENNYKTTKPVLDS |
| | | DGSFFLYSTLTVDKSRWQQGNVFSCSVMHEALHNHYTQKSLSLSPGK |
| 2 | IL15Rα- | ITCPPPMSVEHADIWVKSYSLYSRERYICNSGFKRKAGTSSLTECVLNK |
| | (GGGGS)3-sFc | ATNVAHWTTPSLKCIRGGGGSGGGGSGGGGSAPELLGGPSVFLFPPKP |
| | | KDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKG |
| | | QPREPQVYTLPPSRDELTKNQVSLRCHVKGFYPSDIAVEWESNGQPEN |
| | | NYKTTKPVLDSDGSFFLYSTLTVDKSRWQQGNVFSCSVMHEALHNHY |
| | | TQKSLSLSPGK |
| 3 | IL-15 mutant | NWVNVISDLKKIEDLIQSMHIDATLYTESDVHPSCKVTAMKCFLLELQ |
| | | VISLESGDASIHDTVENLIILANDSLSSNGNVTESGCKECEELEEKNIKEF |
| | | LQSFVHIVQMFINTS |
| 4 | WT IL-15 | NWVNVISDLKKIEDLIQSMHIDATLYTESDVHPSCKVTAMKCFLLELQ |
| | | VISLESGDASIHDTVENLIILANNSLSSNGNVTESGCKECEELEEKNIKEF |
| | | LQSFVHIVQMFINTS |
| 5 | IL15Rα sushi | ITCPPPMSVEHADIWVKSYSLYSRERYICNSGFKRKAGTSSLTECVLNK |
| | domain | ATNVAHWTTPSLKCIR |
| 6 | sFc | APELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVS |
| | | NKALPAPIEKTISKAKGQPREPQVYTLPPSRDELTKNQVSLRCHVKGFY |
| | | PSDIAVEWESNGQPENNYKTTKPVLDSDGSFFLYSTLTVDKSRWQQG |
| | | NVFSCSVMHEALHNHYTQKSLSLSPGK |
| 7 | GGGGS linker | GGGGS |
| 8 | (GGGGS)3 | GGGGSGGGGSGGGGS |
| | linker | |

### EXAMPLES

### Example 1: Preparation of an Agonistic Interleukin 15 Complex

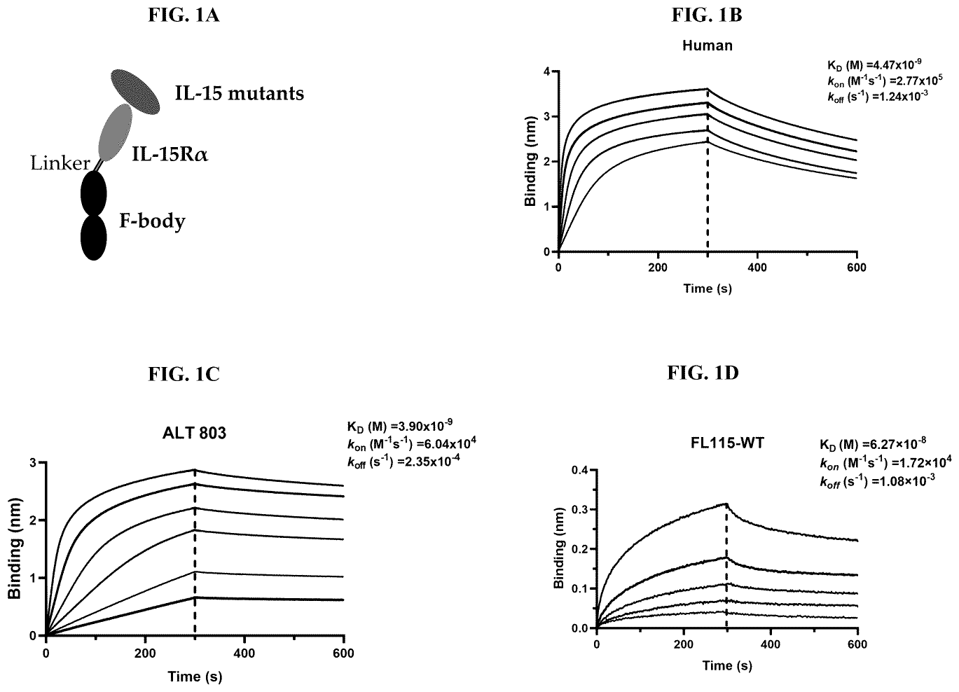

A schematic representation of an agonistic interleukin 15 complex is shown in FIG. 1A. The genes encoding IL15Rα-GGGGS-sFc (SEQ ID NO: 1), IL15Rα-(GGGGS)₃-sFc (SEQ ID NO:2), and IL15 mutants (sequence SEQ ID NO: 3) genes were synthesized by Nanjing GenScript Company (Nanjing, GenScript). The genes were cloned into the eukaryotic expression vector PTT (Thermo Fisher). After sequences of the expression vectors were confirmed, the recombinant proteins were expressed transiently using Expi293 Expression System (Thermo Fisher) according to the manufacturer's instructions and purified by protein G resin (GE Healthcare). Agonistic interleukin 15 complexes were purified from the conditioned culture media and dialyzed into PBS. The purity of agonistic interleukin 15 complex was examined by SDS-PAGE, and protein concentration was measured spectrophotometrically at 280 nm (NanoVue, GE Healthcare) (for specific transformation, expression, and purification methods see: Ying, Tianlei et al. JOURNAL OF BIOLOGICAL CHEMISTRY, 2012. 287(23): 19399-19408).

### Example 2: Binding Activity of Agonistic Interleukin 15 Complexes to IL-15R13

The binding activity of an agonistic interleukin 15 complex to the receptor IL-15R β was assayed using an Octet-RED (Pall ForteBio) Bio-Layer Interferometry (BLI) instrument. Briefly, the IL-15R β protein at 5 μg/mL in kinetics buffer (PBS buffer supplemented with 0.02% Tween 20) was immobilized onto NI-NTA biosensors until saturation (see, for instance, Wang, Chunyu et al. “Engineered Soluble Monomeric IgG1 Fc with Significantly Decreased Non-Specific Binding.” Frontiers in immunology vol. 8 1545. 13 Nov. 2017, PMID: 29181008; Wang, Chunyu et al. “Design of a Novel Fab-Like Antibody Fragment with Enhanced Stability and Affinity for Clinical use.” Small methods vol. 6, 2 (2022): e2100966; and Wang, Chunyu et al. “Design of a Novel Fab-Like Antibody Fragment with Enhanced Stability and Affinity for Clinical use.” Small methods vol. 6, 2 (2022): e2100966, PMID: 35174992). The baseline was established in kinetics buffer and loaded biosensors were dipped into wells containing serial dilutions of an agonistic interleukin 15 complex for 300 s. FL115 (also named FL115-V2), an agonistic interleukin 15 complex according to an embodiment of the application, contains a monomeric IL-15 receptor αSu/Fc fusion protein having the amino acid sequence of SEQ ID NO: 2 and a mutant IL15 having the amino acid sequence of SEQ ID NO: 3. FL115-WT, another agonistic interleukin 15 complex according to an embodiment of the application, contains a monomeric IL-15 receptor αSu/Fc fusion protein having the amino acid sequence of SEQ ID NO: 2 and the wild-type IL15 having the amino acid sequence of SEQ ID NO: 4. The agonistic interleukin 15 complex was bound to the immobilized IL-15R β protein. The binding complexes of the IL-15R β protein-agonistic IL15 complex were then allowed to dissociate in kinetics buffer. Global data fitting to a 1:1 binding model was used to estimate the affinity using Data Analysis software version 8.1. The kon (association rate constant), koff (dissociation rate constant), and KD (equilibrium dissociation constant) values were determined by averaging binding curves within a dilution series having R2 values greater than the 90% confidence level.

The results of the assay are illustrated in FIGS. 1B-1D. Referring now to FIG. 1B-FIG. 1D, the KD of FL115 to IL-15R β was 4.47×10⁻⁹ M, the binding constant was 2.77×105 M⁻¹ s⁻¹, and the dissociation constant was 1.24×10⁻³ s⁻¹, thereby demonstrating similar kinetic and affinity characteristics as compared to the control ALT 803. The results of FIG. 1B-FIG. 1D demonstrate that the IL-15 receptor αSu fused with a Fc monomer did not affect its protein interactions, and the binding activity of FL115 to IL-15R β was comparable to that of ALT 803, an agonistic interleukin 15 complex containing an IL-15 mutant and an IL-15 receptor αSu/Fc fusion protein fused to a Fc dimer.

### Example 3: Cell Proliferation Studies of Agonistic Interleukin 15 Complexes

The biological activity of FL115 was further evaluated using a cell proliferation assay that was performed using CTLL2 cells and M-07e cells (megakaryocytes). CTLL-2 cells are dependent on IL-2 for growth and proliferation, and IL-15 can promote the growth and proliferation of CTLL-2 cells. Therefore, CTLL-2 can be used as a quantitative assay for IL-15 biological activity. The samples to be tested were added to CTLL-2 cells with an agonistic interleukin 15 complex, cultured for 24 h, and the proliferation of the cells was detected using the CCK-8 (Cell Counting Kit-8) method to analyze their biological activity. Megakaryocytes M-07e are an IL-15-dependent cell line, and thus IL-15 can stimulate their proliferation (see, for instance, Meazza, R et al. “Interleukin (IL)-15 induces survival and proliferation of the growth factor-dependent acute myeloid leukemia M-07e through the IL-2 receptor beta/gamma.” International journal of cancer vol. 78, 2 (1998): 189-95, PMID: 9754651; and Finch, D K et al. “Identification of a potent anti-IL-15 antibody with opposing mechanisms of action in vitro and in vivo.” British journal of pharmacology vol. 162, 2 (2011): 480-90, PMID: 20942844). FL115-V1, another agonistic interleukin 15 complex according to an embodiment of the application, contains a monomeric IL-15 receptor αSu/Fc fusion protein having the amino acid sequence of SEQ ID NO: 1 and a mutant IL15 having the amino acid sequence of SEQ ID NO: 3. M-07e cell lines in logarithmic growth phase were washed once with 20 mL of PBS, and then 50 μL of cell suspension was added to each well of a 96-well white plate using experimental medium and adjusting the cell concentration to 2.0×10⁵ cells/mL. 50 μL of diluted FL115 (starting point working concentration 1.2 μg/mL) was added to each well of a 96-well sample plate and mixed with M-07e cells. 2 replicate wells were made for each concentration gradient point. The 96-well white plates were incubated for 48 hours at 37° C. in a 5% CO₂ incubator. Detection of fluorescent signals with the CellTiter-Glo Luminescent Cell Viability Assay kit. The luminescence signal value is detected using a microplate reader.

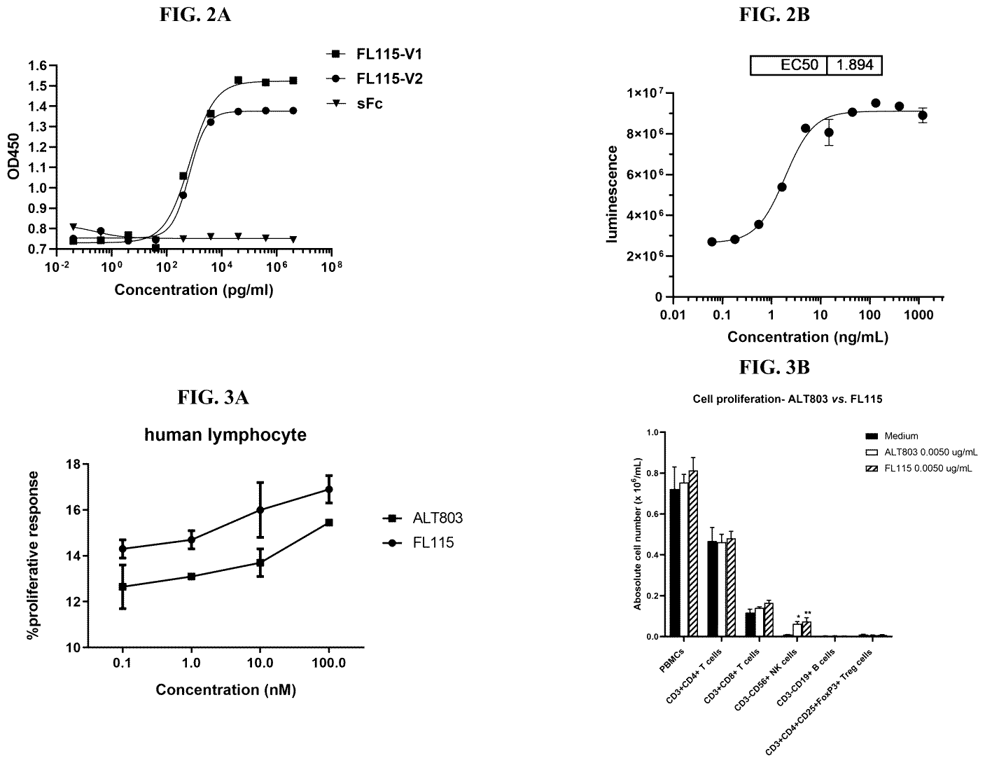

The results of the assay are illustrated in FIG. 2A-FIG. 2B. Shown in FIG. 2A are the proliferative activity of CTLL2 cells in samples treated with FL115-V1 and FL115-V2. As shown in FIG. 2B, FL115 significantly stimulated the proliferation of M-07e cells in a dose-dependent manner. It was noted that the higher the protein concentration used in an assay, the more pronounced the proliferation of M-07e cells observed. The EC50 was 1.894 ng/ml. The assays of the present example provided another demonstration of the biological activity of an agonistic interleukin 15 complex of the application in an in vitro assay.

### Example 4: Immunostimulatory Effects of FL115 on Human Immune Cells

To assess the FL115 mediated responses of human immune cells, studies were conducted with human PBMCs incubated with FL115. For proliferation assays, PBMC proliferation was detected using CFSE. CFSE labeled PBMC cells were stimulated in 2e5 plates per well at concentrations of 100 nM, 10 nM, 1 nM, and 0.1 nM of FL115 and ALT 803 for stimulation, respectively. Cells were incubated for 4 days and then analyzed by flow cytometry to determine cell proliferation based on CFSE. For assessment of immune cell subset and activation markers, human PBMCs were cultured in various concentrations of FL115 and ALT 803, stained under appropriate conditions with marker-specific antibodies, and analyzed on a FACSVerse flow cytometer (BD Biosciences) using FACSuite software.

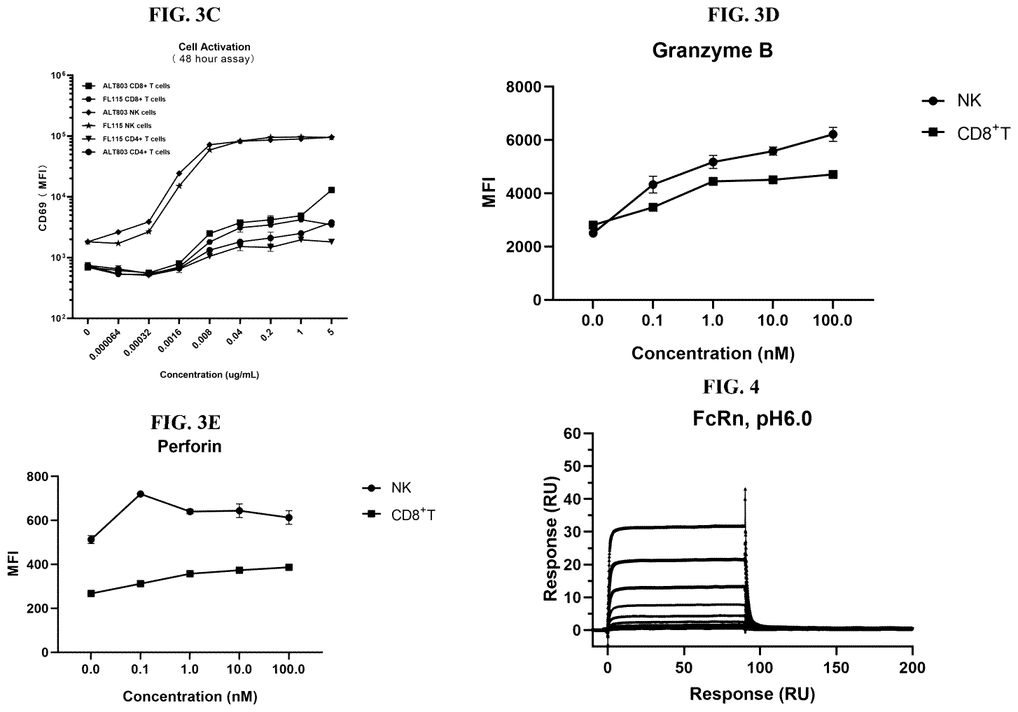

Referring now to FIG. 3A-FIG. 3E, FL115 dose-dependent lymphocyte proliferation was observed in human PBMC cultures, and better than ALT 803 overall (FIG. 3A). Treatment with 0.0050 ug/ml FL115 resulted in pronounced proliferation in NK counts, significantly better than ALT 803 (FIG. 3B). In addition, cell surface activation marker expression of CD69 on NK, CD8+ T cells and CD4+ T cells was stimulated by FL115 in a dose-dependent manner (FIG. 3C). FL115 also induced increased granzyme B and perforin expression in both human NK cells and CD8+ T cells (FIG. 3D and FIG. 3E). Together, these findings indicate that FL115 at a concentration as low as 0.01 nmol/L is capable of activating human immune cells in vitro.

### Example 5: BLI Studies of FL115 and FcRn

In the present example, the binding of FL115 to human FcRn was measured using SPR using standard assay protocol for SPR See, for instance, Ying, Tianlei et al. “Soluble monomeric IgG1 Fc.” The Journal of biological chemistry vol. 287, 23 (2012): 19399-408, PMID: 22518843; and Ying, Tianlei et al. “Monomeric IgG1 Fc molecules displaying unique Fc receptor interactions that are exploitable to treat inflammation-mediated diseases.” mAbs vol. 6, 5 (2014): 1201-10, PMID: 25517305). Ligand coupling was performed using the CM5 chip, referring to the operating instructions provided with the amino coupling kit. PBS-P+ was used as the buffer. Flow cell (Fc) 1 and Flow cell 2 flow channels were activated with 11.5 mg/ml NHS and 75 mg/ml EDC (1:1) for 420 s. The ligand was diluted to 1 μg/mL with NaAc pH 5.0 and then coupled to the activated flow channel Fc 2 at a flow rate of 10 μL/min, with the coupling level set at 100 RU. The Fc 1 and Fc 2 flow channels were closed by injection of 1 M ethanolamine for 420 s. The binding of Human FcRn to FL115 under different pH conditions was assayed using PBS-P+ at pH 6.0 and pH 7.4 as buffers, respectively. FL115 was diluted to 200 μg/mL with PBS-P+ (pH 6.0 or pH 7.4) buffer and this was used as the highest concentration. The 200 ug/mL of FL115 was diluted twofold with PBS-P+ (pH 6.0 or pH 7.4) buffer up to 0.78 μg/mL. The flow rate was set at 30 μL/min and the two-fold gradient of FL115 was sequentially injected into the flow channel and bound to the Human FcRn coupled to the chip with a binding time of 90 s and a dissociation time set at 110 s. After dissociation was complete, regeneration was performed with PBS-P+ at pH 7.4. Binding, dissociation, and regeneration were repeated for different concentrations of FL115. Finally, the data were analyzed using the Steady State Affinity model selected by Biacore T200 evaluation software to detect the binding rate of FL115 and human FcRn. FcRn was diluted to a fixed concentration of 1 μg/mL with NaAc pH 5.0 and FL115 diluted in a twofold gradient with PBS-P+ (pH 6.0 or pH 7.4) buffer was used as the analyte for detection.

Referring now to FIG. 4, the affinity KD value of FL115 to FcRn was 4.07×10⁻⁶ M at pH 6.0, and it did not bind FcRn at pH 7.4 (data not shown). The above results indicated that the long-acting agonistic IL-15 complex FL115 maintained the characteristic pH-dependent FcRn binding of sFc, which was indicative of its extended in vivo half-life.

### Example 6: Anti-Tumor Effect of FL115 in MC38 Subcutaneous Transplantation Tumor Model

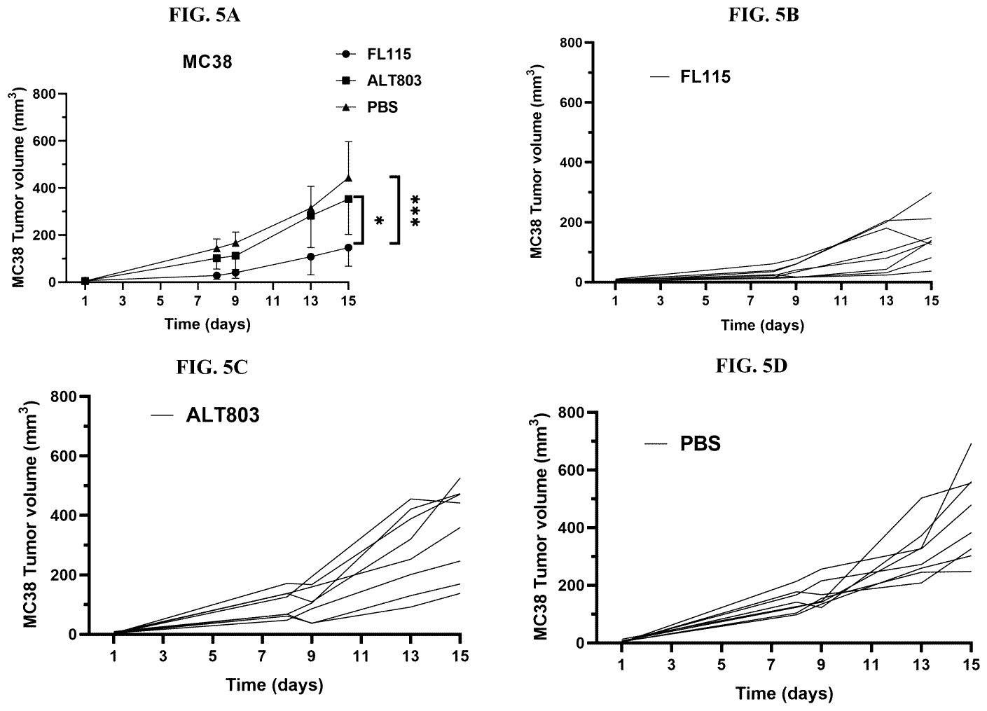

In the present example, the anti-tumor effect of FL115 was evaluated in an MC38 (a mouse colon adenocarcinoma cell line) subcutaneous transplantation tumor model. Briefly, 5×10⁵ MC38 mouse colon cancer cells were injected on the right side of the back in 6-8 week old female C57BL/6 mice (purchased from Shanghai Jihui Experimental Animal Co., Ltd.). FL115 at 0.2 mg/kg, positive control (ALT803), and negative control (PBS) were administered starting on days 1 and 8 after inoculation. The tumor size and weight of the mice were measured on the indicated days (see FIG. 5A-FIG. 5E).

Referring now to FIG. 5A-FIG. 5E, in the MC38 subcutaneous transplantation tumor model, FL115 and ALT803 both had anti-tumor efficacy with statistically significant differences from the negative control group. Notably, the anti-tumor effect of FL115 was significantly better than the positive control ALT803. Additionally, the increase in body weights of the mice over time (FIG. 5E) further confirmed the safety of FL115.

### Example 7: Detection of Tumor Microenvironmental Changes Induced by FL115 in the MC38 Subcutaneous Tumor Model

In the present example, the changes of immune cells in tumor tissues in MC38 subcutaneous tumor model were evaluated. 5×10⁵ MC38 tumor cells were inoculated subcutaneously into C57 mice and randomized into groups on the day after inoculation. Each group of 8 mice was administered FL115 and ALT803 intravenously at a dose of 0.2 mg/kg every 7 days for a total of three doses. The mice were executed 24 h after the third dose, and the tumor tissue was peeled off. The tumors were gently broken into smaller pieces with forceps and digested with 0.2 mg/ml Collagenase IV (Sigma-Aldrich) and 0.1 mg/ml DNAse I (Sigma-Aldrich) for 1 h at 37° C. Single cell suspensions were collected, filtered through a 70 μm nylon mesh and mouse lymphocytes were isolated using Mouse Lymphocyte Isolation Solution (Dacor for). Lymphocytes were stained with flow antibody (purchased from BD) for surface proteins and fixed broken membranes for intracellular proteins (purchased from Sigma-Aldrich) and immediately put on a flow cytometer (purchased from BD) to detect CD8 T cells and NK cells. The results are shown in the figure. Flow assay results for tumors stripped from one mouse are shown on the left, and the proportion of immune cells (mean±SD) measured in each group of mice is shown on the right, where one point represents a tumor in one mouse.

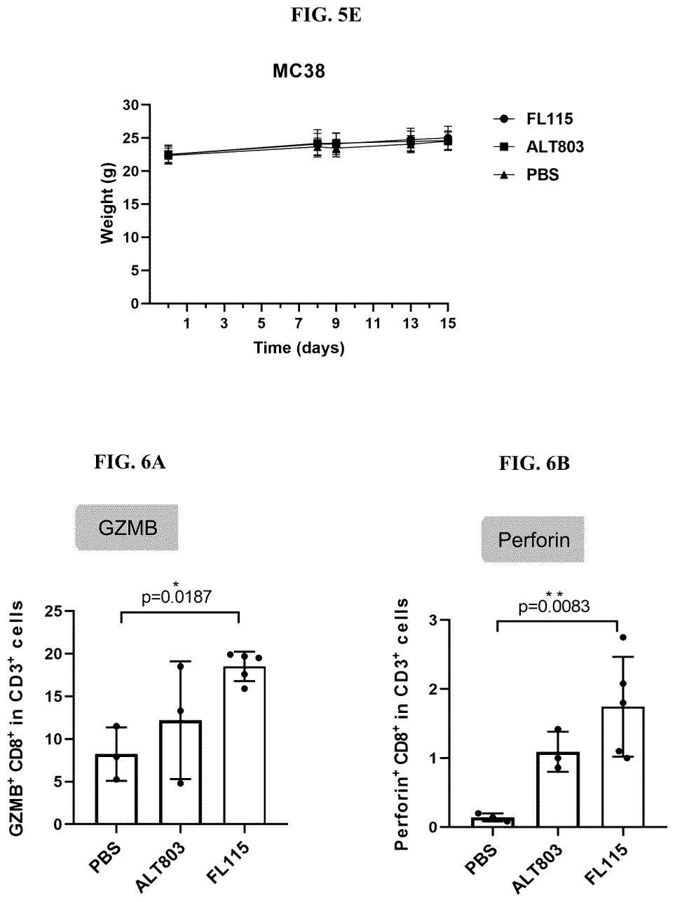

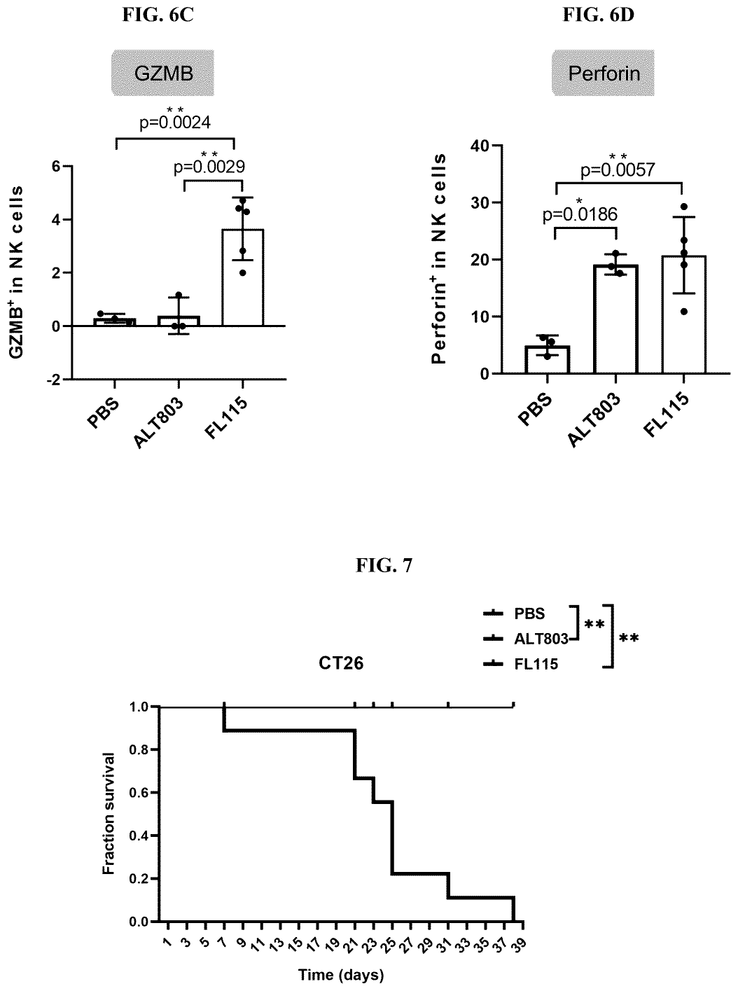

Referring now to FIG. 6A-FIG. 6D, at the same administered dose (0.2 mg/kg), FL115 induced more granzyme secretion from tumor-infiltrating killer NK cells and CD8+ T cells than the positive control ALT803 (NK: FL115 (3.65%) versus PBS (0.30%), P<0.01; CD8+T: FL115 (18.52%) versus PBS (8.23%), P<0.05) and more perforin+CD8+ in CD3+ cells than the positive control ALT803 (NK: FL115 (20.78%) vs. PBS (4.99%), P<0.01; CD8+T: FL115 (1.75%) vs. PBS (0.14%), P<0.01) (see FIG. 6A-FIG. 6D). The above results demonstrated the stronger antitumor activity of FL115 compared to the positive control ALT803, as FL115 antitumor treatment resulted in a stronger killing function of NK cells and CD8+ T cells than ALT803 in the tumor microenvironment. Additionally, the effect of FL115 on NK cell activation was significantly different as compared to ALT803.

### Example 8: Anti-Tumor Effect of FL115 in CT26 Metastatic Tumor Model

In the present example, FL115 was evaluated in a CT26 metastatic tumor model. Briefly, 2×10⁵ CT26 mouse colon cancer cells were injected in the tail vein of 6-8 week old male C57BL/6 mice (purchased from Shanghai Jihui Experimental Animal Co., Ltd.). The administration of 0.2 mg/kg of FL115, positive control (ALT803), and negative control (PBS) was started on days 1, 4, 8, and 11 after the injection.

Referring now to FIG. 7, in the CT26 mouse colon cancer metastasis tumor model, FL115 had clear anti-tumor efficacy, whereas all mice in the negative control group died during the observation period. It was additionally observed that the mice in the FL115 group had good vital signs.

### Example 9: Anti-Tumor Effect of FL115 in Combination with Anti-PD-1 Antibody in Mice

In the present example, the anti-tumor effect of FL115 in combination with an anti PD-1 antibody (InVivoPlus anti-mouse PD-1 (CD279), Clone: RMP1-14, Catalog:BP0146-100 mg) was evaluated (see, for instance, Li, Howard Y et al. “The Tumor Microenvironment Regulates Sensitivity of Murine Lung Tumors to PD-1/PD-L1 Antibody Blockade.” Cancer immunology research vol. 5, 9 (2017): 767-777, PMID: 28819064). In particular, the ability of FL115 to activate NK cells to enhance antitumor effects in patients with tumors that do not respond to PD1 monoclonal antibodies was measured. Furthermore, the anti-tumor activity of FL115 in combination with PD1 antibody in a non-small cell lung cancer (NSCLC) model to test whether the two have synergistic effects. 5×10⁵ lung adenocarcinoma CMT167 cells or LLC cells were injected on the right side of the back in 6-8 week old male C57BL/6 mice (purchased from Shanghai Jihui Experimental Animal Co., Ltd.) (see, for instance, Li, Howard Y et al. “The Tumor Microenvironment Regulates Sensitivity of Murine Lung Tumors to PD-1/PD-L1 Antibody Blockade.” Cancer immunology research vol. 5, 9 (2017): 767-777, PMID: 28819064; Tang, Honglin et al. “Inhibition of COX-2 and EGFR by Melafolone Improves Anti-PD-1 Therapy through Vascular Normalization and PD-L1 Downregulation in Lung Cancer.” The Journal of pharmacology and experimental therapeutics vol. 368, 3 (2019): 401-413, PMID: 30591531; and Johnson, Amber M et al. “Cancer Cell-Intrinsic Expression of MHC Class II Regulates the Immune Microenvironment and Response to Anti-PD-1 Therapy in Lung Adenocarcinoma.” Journal of immunology (Baltimore, Md.: 1950) vol. 204, 8 (2020): 2295-2307, PMID: 32179637). Animals in each group were given FL115, anti-PD1 antibody and FL115 combined with anti-PD1 antibody, while a lysis control group (PBS) was set up. Each subject was administered intravenously at 10 mg/kg for FL115 and 200 μg for anti-PD1 antibody, FL115 was administered once a week for a total of 3 doses (D1, D8, D15) and anti-PD1 antibody was administered 3 times (D11, D14, D17).

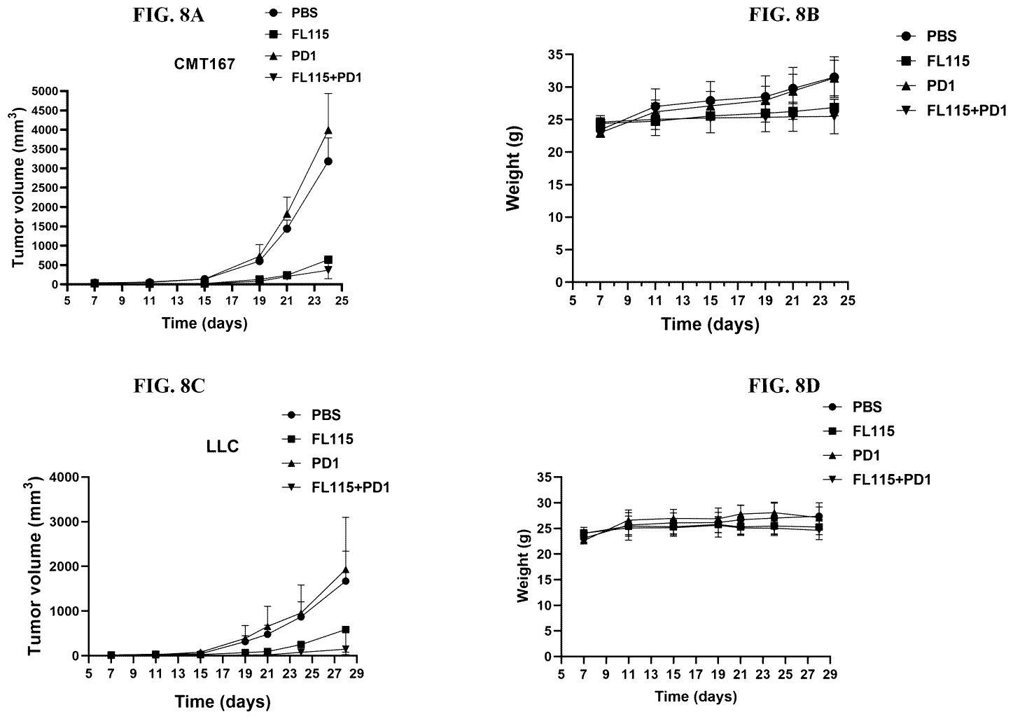

Referring now to FIG. 8A-FIG. 8D, for the lung adenocarcinoma CMT167 cell subcutaneous transplantation tumor C57 mouse model, the relative tumor TGI of FL115 combined with PD-1 monoclonal antibody administration group was 88.43% (P<0.0001) after 24 day time point of administration compared to the negative control group, and no animals were found to have complete tumor regression and no individual animals in complete remission. After administration of FL115 at 10 mg/kg, the relative tumor growth inhibition (TGI) at Day 24 was 79.90% (P<0.001) compared with the negative control (PBS) group, and one mouse had complete tumor growth inhibition; after administration of PD1 monoclonal antibody at 200 μg, the relative TGI at Day24 was −25.37% (P>0.05) compared with the negative control group. FL115 combined with PD-1 monoclonal antibody had significantly better tumor inhibition as the FL115 combined with PD-1 monoclonal antibody administration group was significantly better than that of the PD1 monoclonal antibody group, with a significant difference (P<0.0001). FL115 combined with PD-1 monoclonal antibody administration group had better tumor inhibition than the FL115 group, although the difference is not significant. For the murine lung cancer LLC cell subcutaneous transplantation tumor C57 mouse model, the relative tumor TGI of FL115 combined with PD-1 monoclonal antibody administration group was 91.12% (P<0.01) compared with the negative control group after Day 28 of administration, and the tumor growth was completely inhibited in two mice. After FL115 administration at 10 mg/kg, the relative TGI was 64.91% compared with the negative control group at Day 28. After FL115 was administered at 10 mg/kg, the relative TGI at Day 28 was 64.91% (P<0.05), and one mouse was completely inhibited. After PD1 monoclonal antibody was administered at 200 μg, the relative TGI at Day2 8 was −15.79% (P>0.05) compared with the negative control group, and one mouse died at Day 24. The tumor suppressive effect of FL115 combined with PD-1 monoclonal antibody administration group was significantly better than that of PD1 monoclonal antibody group with a significant difference (P<0.05); the tumor suppressive effect of FL115 combined with PD-1 monoclonal antibody administration group was better than that of FL115 group, but there was no significant difference. None of the animals in all dosing groups showed significant weight loss and no morbidity during the studies.

Taken together, the results indicated that the combination of FL115 with anti-PD-1 antibody significantly reduced tumor load and was effective against both CMT167 cell (a highly metastatic subclone of murine alveogenic lung carcinoma cell line CMT 167) and Lewis lung carcinoma cell (LLC) transplanted tumors.

### Example 10: BLI Studies of the Affinity of FL115 for CD16a 158V and CD16a 158F

In the present example, the affinity of FL115 for CD16a 158V and CD16a 158F was measured using BLI. Monomeric Fc, which had previously been tested, does not bind to CD16a and CD16b. In general, CD16a acts as a receptor for the activation of innate immune cells with killing function, such as NK cells. There are two polymorphic variants of the receptor: 158V, which has a high affinity for IgG1, and 158F, which has a low affinity for IgG1. Based on the foregoing, the affinity of FL115 for the Fc receptors CD16a V158 and F158 was evaluated. Briefly, the CD16a 158F and 158V protein at 5 μg/mL in kinetics buffer (PBS buffer supplemented with 0.02% Tween 20) was immobilized onto NI-NTA biosensors until saturation (see, for instance, Wang, Chunyu et al. “Engineered Soluble Monomeric IgG1 Fc with Significantly Decreased Non-Specific Binding.” Frontiers in immunology vol. 8 1545. 13 Nov. 2017, PMID: 29181008; and Wang, Chunyu et al. “Design of a Novel Fab-Like Antibody Fragment with Enhanced Stability and Affinity for Clinical use.” Small methods vol. 6, 2 (2022): e2100966, PMID: 35174992). The baseline was established in kinetics buffer and loaded biosensors were dipped into wells containing serial dilutions of FL115 and variants for 300 s. CD16a antigen-FL115 complexes were then allowed to dissociate in kinetics buffer. Global data fitting to a 1:1 binding model was used to estimate the affinity using Data Analysis software version 8.1. The kon (association rate constant), koff (dissociation rate constant), and KD (equilibrium dissociation constant) values were determined by averaging binding curves within a dilution series having R2 values greater than the 90% confidence level.

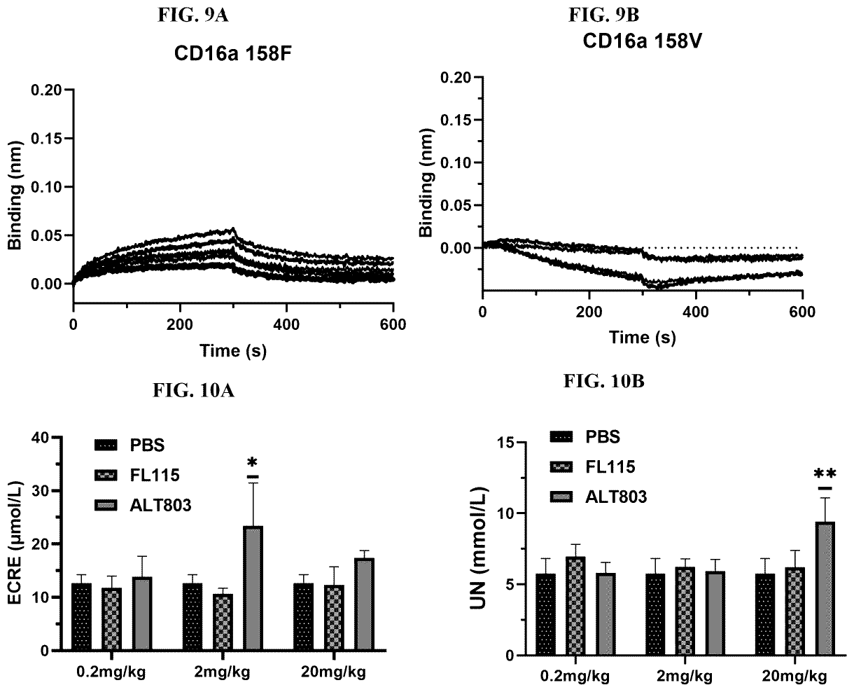

Referring now to FIG. 9A-FIG. 9B, FL115 did not bind to either CD16a 158V nor CD16a 158F, indicative of the low toxicity of FL115.

### Example 11: Safety Evaluation of FL115

In the present example, the potential toxicity of FL115 and ALT 803 in BALB/C mice was evaluated by administering different doses (0.2 mg/kg, 2 mg/kg, 20 mg/kg) of FL115 and ALT 803 in tail vein for 4 weeks (once a week, 4 times).

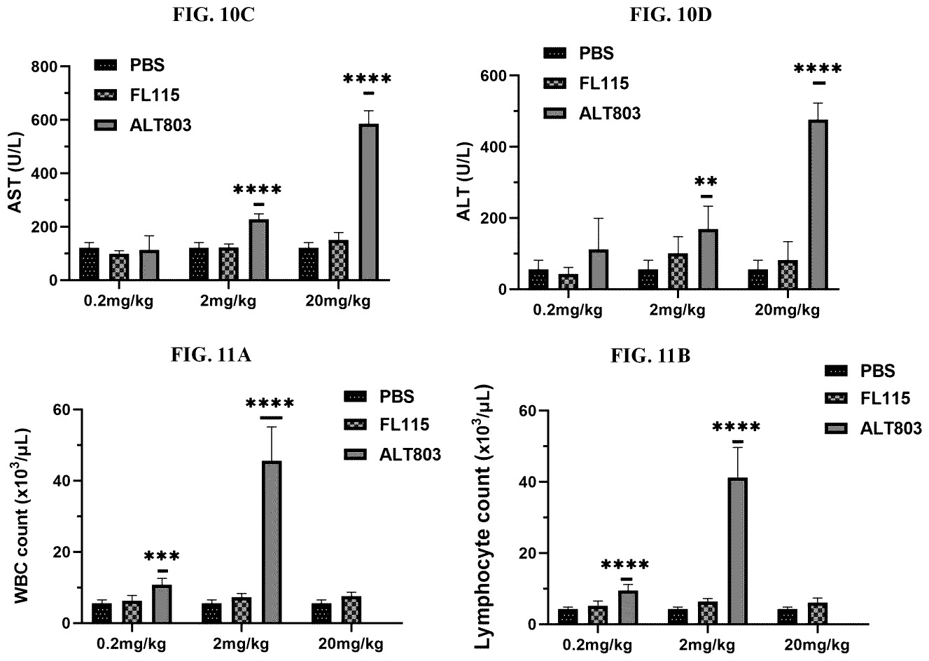

Referring now to FIG. 10A-FIG. 10D, the results demonstrated that ALT 803 caused a significant increase in liver function indicators glutamate transaminase (ALT) and glutathione transaminase (AST) as compared to the control group at doses of 2 mg/kg and 20 mg/kg (ALT: 2 mg/kg P<0.0001, 20 mg/kg P<0.0001; AST: 2 mg/kg P<0.0001, 20 mg/kg P<0.0001). 0.0001, 20 mg/kg P<0.0001) and significantly higher compared to the FL115 group (ALT:2 mg/kg P<0.0001, 20 mg/kg P<0.0001; AST: 2 mg/kg P>0.05, 20 mg/kg P<0.0001), both suggesting substantial liver damage. Furthermore, ALT 803 at a dose of 2 mg/kg caused a significant increase in creatinine (ECRE), an indicator of renal function, compared to the FL115 group (P<0.05), and at a dose of 20 mg/kg caused a significant difference in urea nitrogen (UN), an indicator of renal function, compared to both the control and FL115 groups (P<0.01). These results showed that ALT 803 at 2 and 20 mg/kg caused substantial impairment of liver function, but did not have a significant effect on renal function, whereas FL115 did not have a significant effect on either the liver or the kidneys.

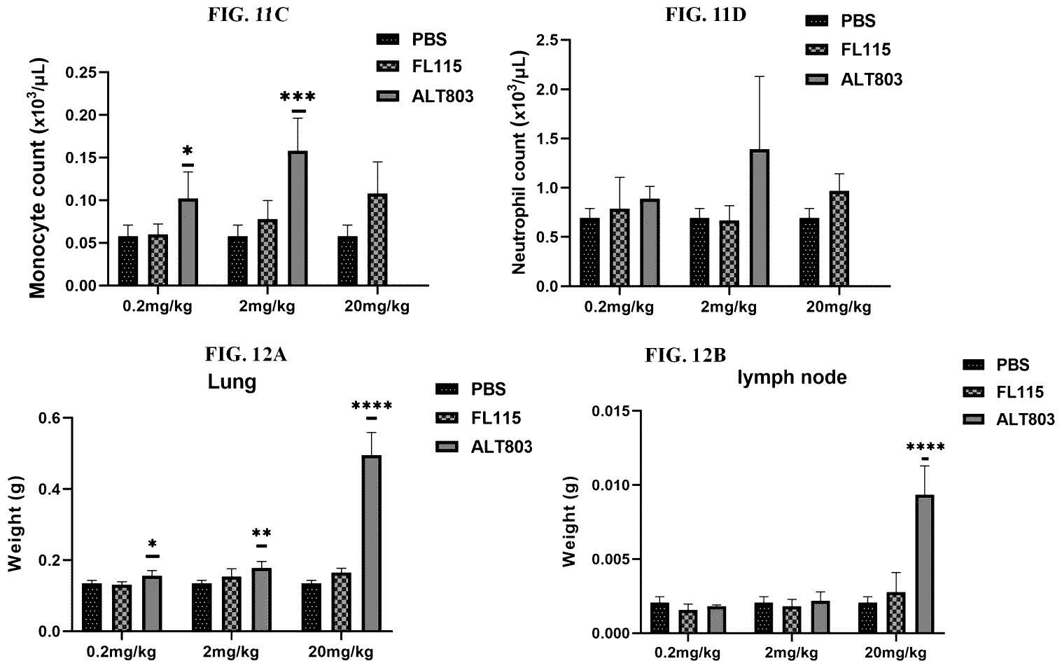

Referring now to FIG. 11A-FIG. 11D, ALT 803 at 20 mg/kg caused all mice to die after day 3, and at 0.2 and 2 mg/kg caused a large increase in leukocytes in mice, which was significantly different from the leukocyte release caused by the control group (WBC: 0.2 mg/kg P<0.001; 2 mg/kg P<0.0001); and from the FL115 group (WBC: 0.2 mg/kg P<0.01; 2 mg/kg P<0.0001). The doses of 0.2 and 2 mg/kg caused a significant increase in lymphocytes in mice, which was significantly different from the lymphocyte release caused by the control group (Lymphocyte: 0.2 mg/kg P<0.0001; 2 mg/kg P<0.0001); and significantly different from the lymphocyte release caused by the FL115 group (Lymphocyte: 0.2 mg/kg P<0.001; 2 mg/kg P<0.0001). The doses of 0.2 and 2 mg/kg caused a large increase in monocytes in mice, which was significantly different from the monocyte release induced by the control group (Lymphocyte: 0.2 mg/kg P<0.05; 2 mg/kg P<0.001); and significantly different from the monocyte release induced by the FL115 group (Lymphocyte: 0.2 mg/kg P<0.05; 2 mg/kg P<0.01).

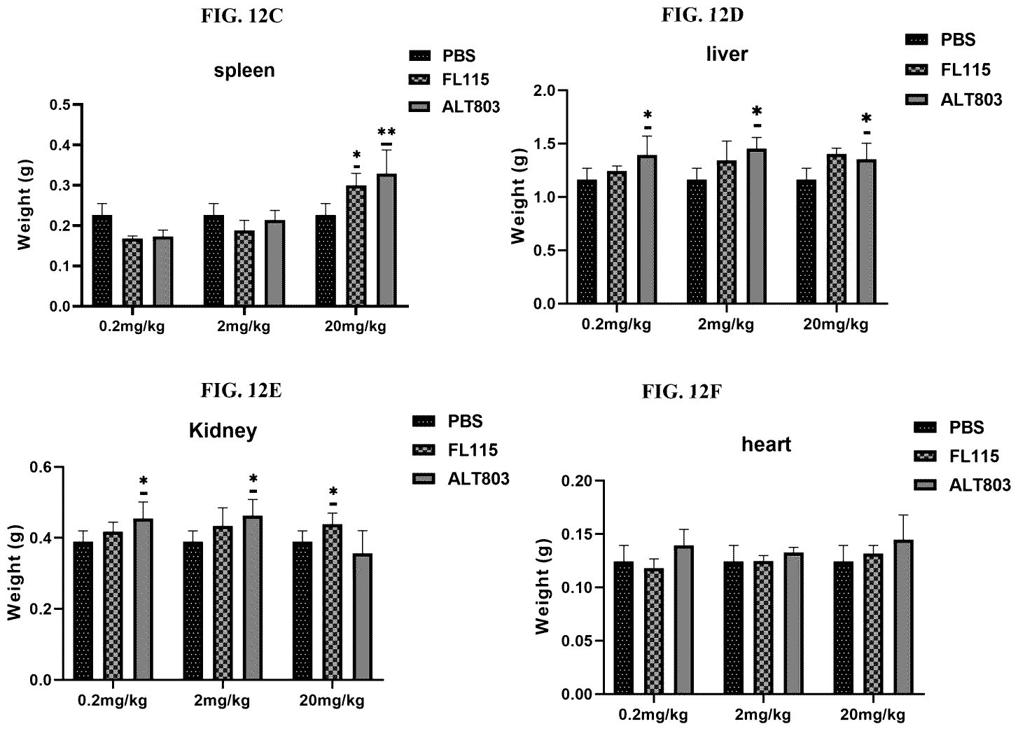

Referring now to FIG. 12A-FIG. 12F, different from the ALT 803 administration group, no significant increases in lung, spleen and lymph node weights or significant increases in visceral weight in comparison to the control group were observed in mice treated with FL115 executed on day 4 after the fourth dose and weighed.

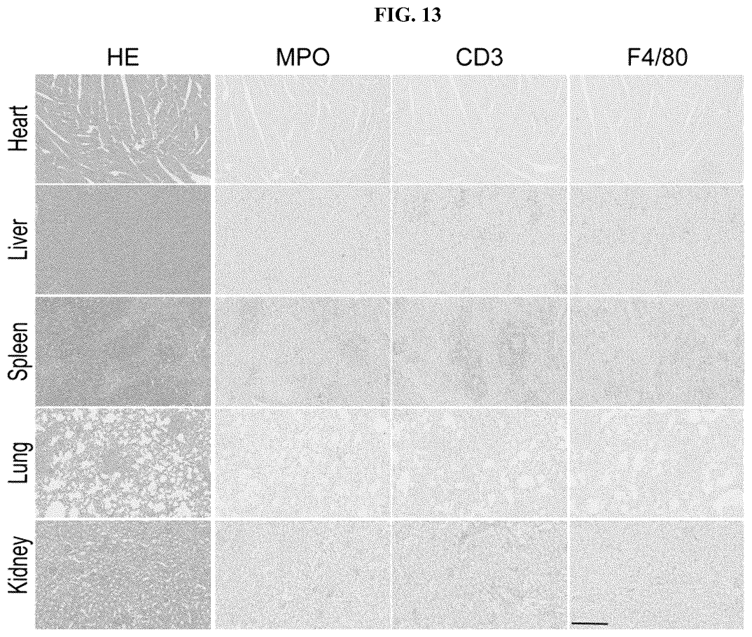

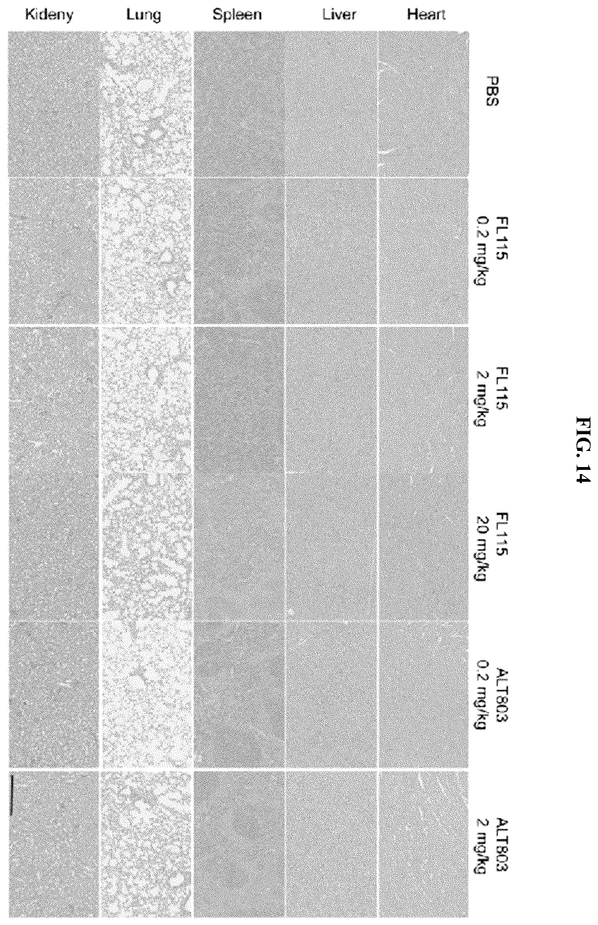

Referring now to FIG. 13, in the ALT 803 20 mg/kg administration group, mice showed no necrosis and mild inflammation in the heart; mild necrosis and severe inflammation in the liver; severe necrosis and severe inflammation in the spleen; moderate necrosis and severe inflammation in the lung; and mild necrosis and severe inflammation in the kidney. FIG. 14 further shows that the hearts of ALT 803 2 mg/kg mice were mildly necrotic in the other administration groups and mildly inflammatory in all other groups. Notably, there was no necrosis in liver and spleen, mild inflammation in FL115 20 mg/kg mice, whereas there was mild inflammation in ALT 803 2 mg/kg. The lungs were free of necrosis and mildly inflamed. No necrosis was observed in the kidneys, and no inflammation was observed in any of them. FL115 showed mild inflammation in liver and spleen except in the high dose group of 20 mg/kg, and basically no significant effect in the low and medium dose groups (0.2 mg/kg and 2 mg/kg), which again supports the high safety of FL115 and confirms no significant toxic side effects at the animal level with increased dose.

Based on the above results, the final tolerable dose of FL115 in mice was determined to be >20 mg/kg, while ALT 803 showed significant toxic side effects when administered at 2 mg/kg.

### Example 12: Effect of FL115 on In Vivo Secretion of Cytokines in Mice

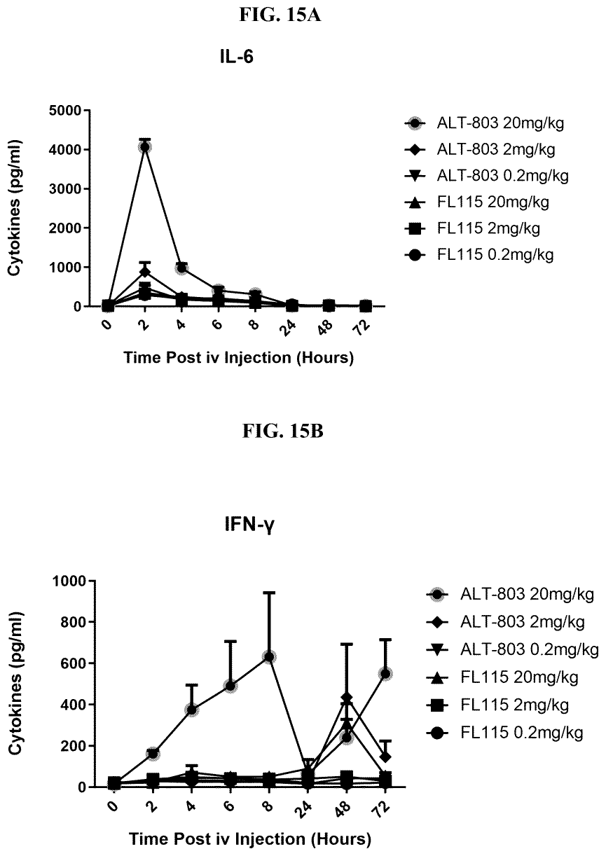

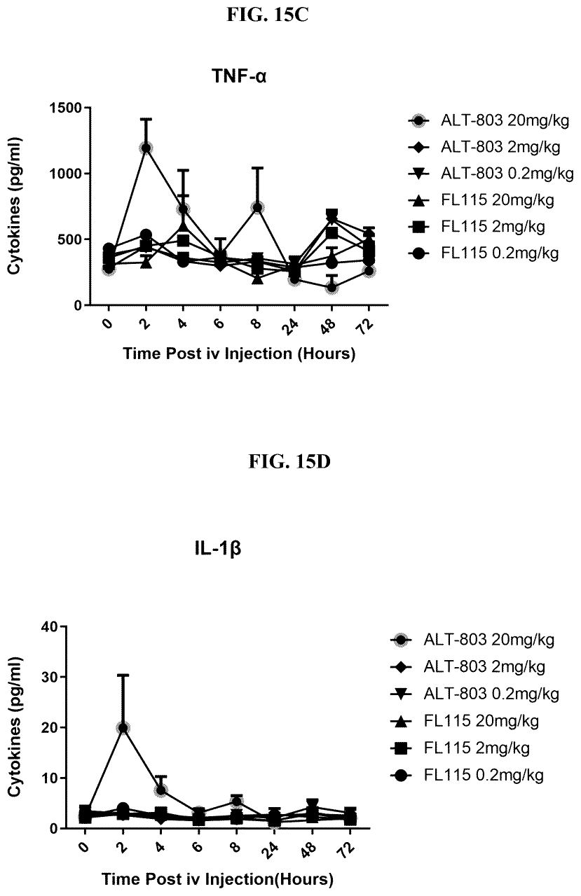

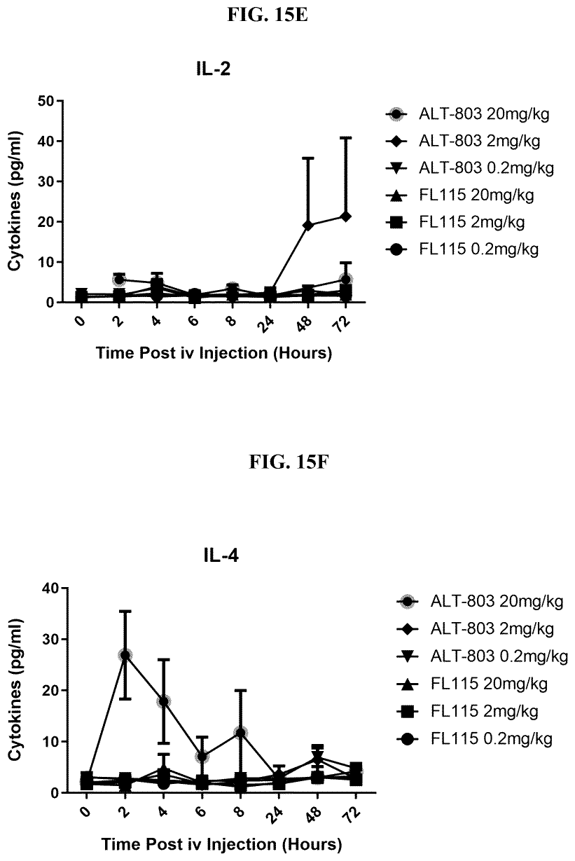

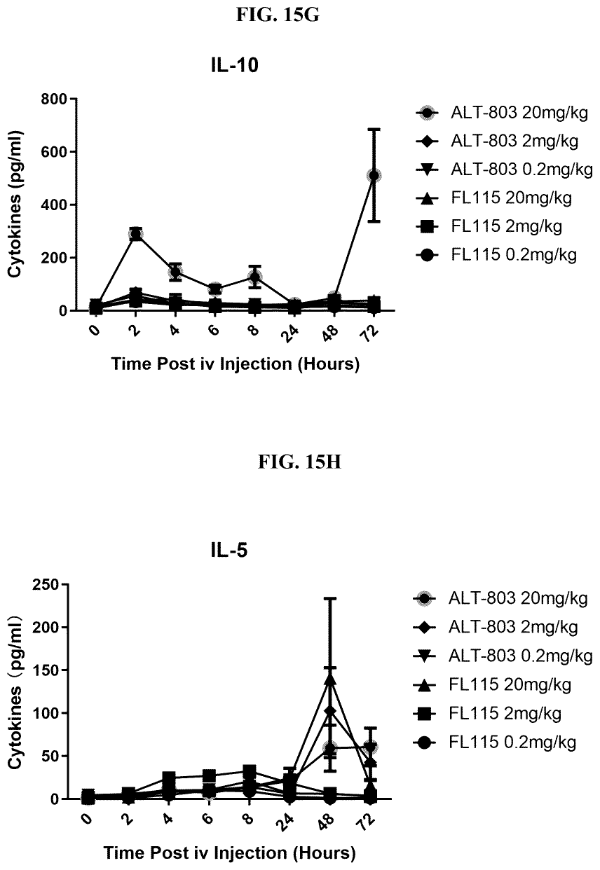

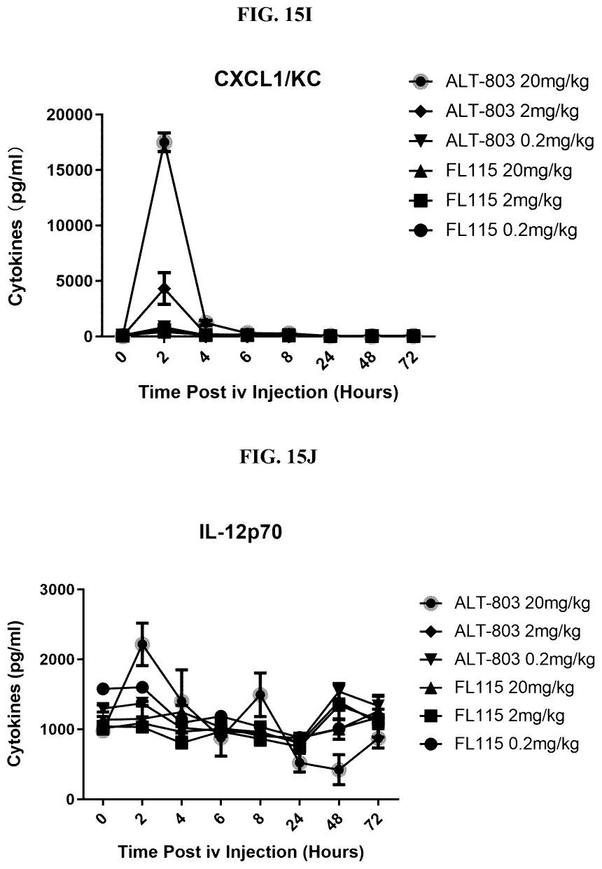

In the present example, the effect of FL115 on the release of ten cytokines, including IFN-γ, IL-10, IL-12p70, CXCL1/KC, IL-1β, IL-2, IL-4, IL-6, IL-5 and TNF-α, was evaluated in mice for the potential risk of inducing a cytokine storm. The assays used chemiluminescence technology based on the Luminex platform. Briefly, six-week-old mice were injected with high and low doses of FL115 intravenously or administered a dose of ALT803 as a control. The time points of blood collection were set at 0 h, 2 h, 4 h, 6 h, 8 h, 24 h, 48 h, and 72 h. The collected mouse sera were analyzed to detect the expression of the above ten cytokines by chemiluminescence detection technology from Shanghai Uninvest Biotechnology Co. The graphs were made using Graphpad software, and the levels of each cytokine secreted in vivo by FL115 and control stimulated mice are shown in FIG. 15A-FIG. 15J.

Referring now to FIG. 15A-FIG. 15J, the results showed that different doses of FL115 had no significant effect on the in vivo release of cytokines IL-1β, IL-2, IL-4, IL-10, IL-5, CXCL/KC and IL-12P/p70 in mice, and that FL115 had a certain degree of effect on the in vivo release of cytokines IL-6, IFN-γ and TNF-α in mice, among which IL-6 was slightly increased after FL115 administration. The release of IL-6 increased slightly after 2 h of FL115 administration, and gradually returned to normal after 24 h. The release of IFN-γ increased slightly after 2 h of FL115 administration, with the highest release in the high-dose 20 mg/kg group at 48 h. The release of TNF-α increased slightly after 2 h of FL115 administration, and gradually decreased to normal level after 6 h.

Further referring to FIG. 15A-FIG. 15J, the effect of different doses of ALT 803 on the release of cytokines in mice was most obvious in the high dose group, in which the release of IL-6, IFN-γ, TNF-α, IL-1β, IL-4, IL-10 and CXCL/KC was the most significant, triggering a cytokine storm that led to the death of mice in this dose group at 96 h after administration. The effect on IL-6, IFN-γ, IL-2 and CXCL/KC release was also significant in the middle dose group, but no mice died.

### Example 13: Anti-Tumor Effect of FL115 in MB49 Mouse Bladder Cancer Model

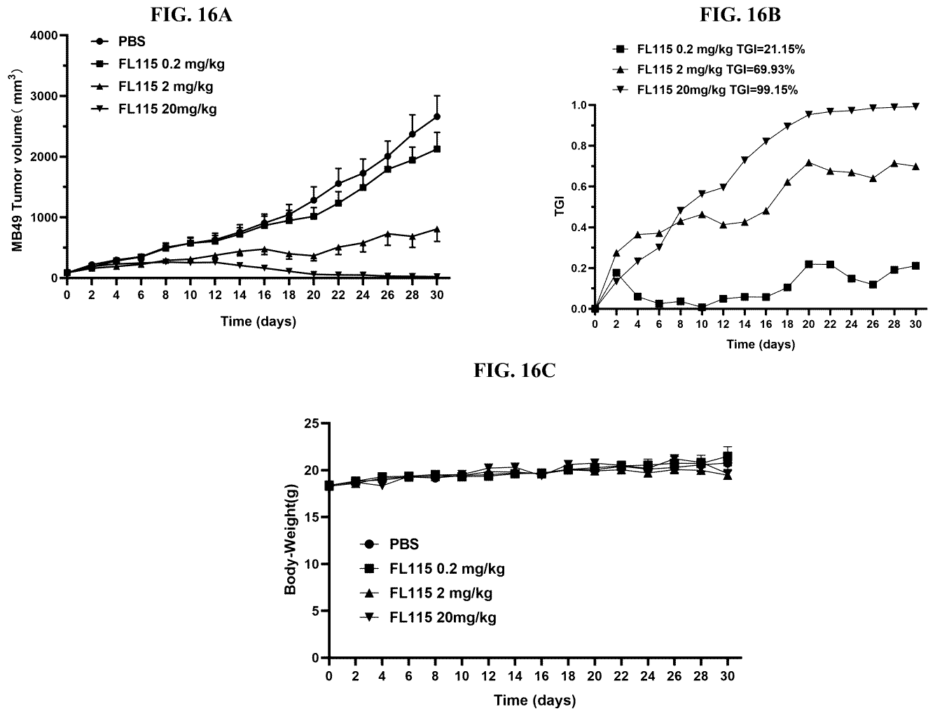

In the present example, the anti-tumor effect of FL115 was evaluated in an MB49 mouse bladder cancer model. Briefly, 1×10⁶ MB49 mouse bladder cancer cells were injected on the right side of the back in 6-8 week old male C57BL/6 mice (purchased from Beijing Vitalihua Laboratory Animal Technology Co., Ltd.). The average tumor volume reached approximately 60-100 mm³ when the mice were administered in groups of 10 mice each, labeled DO. The mice were then administered once a week for a total of 5 doses. The tumor size and weight of the mice were measured on the indicated days (see FIG. 16A-FIG. 16C).

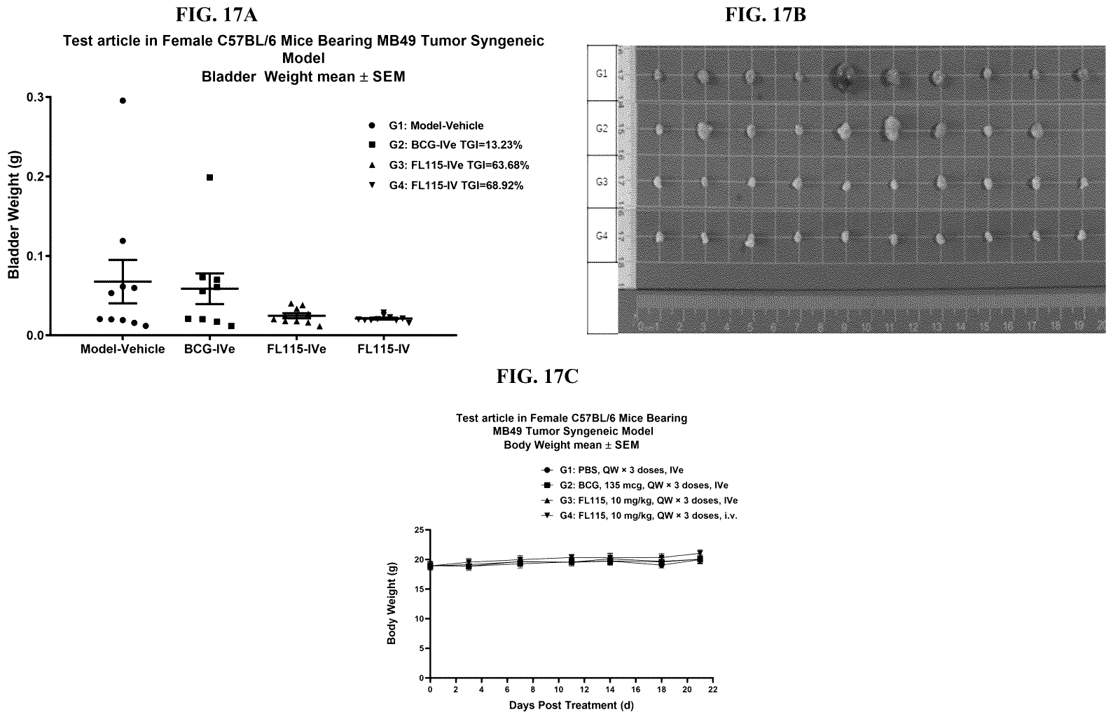

C57BL/6 mice were inoculated with MB49 (a urothelial carcinoma line) cells in situ. MB49 cells were collected at logarithmic growth stage (3rd-4th generation after resuscitation), the culture fluid was removed and washed twice with DPBS before inoculation (cell viability was measured before and after inoculation), and the bladder was pretreated with bladder perfusion pretreatment solution before inoculation. On day 7 after inoculation in situ, i.e., D7, the cells were randomly grouped according to body weight and administered on the day of grouping. Thereafter, the drug was administered once a week for a total of 4 times. The mice were weighed and their status was observed on the indicated dates. The bladders of tumor-bearing mice were weighed and photographed at the end of the experiment (see FIG. 17A-FIG. 17C).

Referring now to FIG. 16A-FIG. 16C, in a subcutaneous tumor-bearing model of mouse bladder cancer, FL115 showed a highly significant dose-dependent effect (FIG. 16A). All mice in the high dose group (20 mg/kg) achieved complete tumor remission, and the tumor inhibition rate in the middle dose group (2 mg/kg) was as high as 69.93% (FIG. 16B). There was no significant change in body weight in all mice, indicating the high safety of FL115 in vivo (FIG. 16C).

Referring now to FIG. 17A-FIG. 17C, in the orthotopic model of bladder cancer in mice, FL115 has a very significant tumor inhibition effect both intravenously and intravesically, and was significantly better than the BCG group. No animals had palpable tumors, i.e., advanced tumors. Compared with the lysate control group, both the bladder perfusion and intravenous FL115 groups significantly reduced tumor load (bladder weight) at Day21, with a 63.68% reduction in mean bladder weight in the bladder perfusion FL115 group and a 68.92% reduction in mean bladder weight in the intravenous FL115 group, both of which had comparable tumor load inhibition; compared with the lysate control group, at Day21, the mean bladder weight of intravesical BCG treatment alone was reduced by 13.23%, indicating that the tumor suppressive effect of FL115 alone, whether administered by bladder instillation or intravenous injection, was significantly better than that of BCG bladder instillation administration alone. (FIG. 17A, B). Secondly, there was no significant change in body weight in all mice (FIG. 17C), indicating the high in vivo safety of FL115 mice.

Based on the above results, it was determined that the risk of FL115 triggering cytokine storm in mice in preclinical studies was low.

### Example 14: Extended Serum Half-Life of FL115

The study aimed to assess the pharmacokinetic profiles of FL115 (e.g., FL115-V2) after single intravenous injection in ICR mice.

In the study, 6- to 8-week male ICR mice (purchased from Beijing Vital River Laboratory Animal Technology Co., Ltd.) were used. Mice were randomized based on body weight and size. In the study, mice were randomized into 4 groups, with 5 animals in each group. The mice of four groups were given a single dose of 0.2 and 2 mg/kg FL115 and positive control ALT803 by intravenous injection, respectively. Serum samples were collected before dosing (0 h), 30 min, 1 h, 2 h, 4 h, 8 h, 10 h, 24 h, 48 h, 72 h and 96 h after dosing and determined for FL115 and ALT803 concentration with the validated ELISA method. Pharmacokinetic parameters of FL115 and ALT803 were calculated with WinNonLin non-compartmental analysis (NCA).

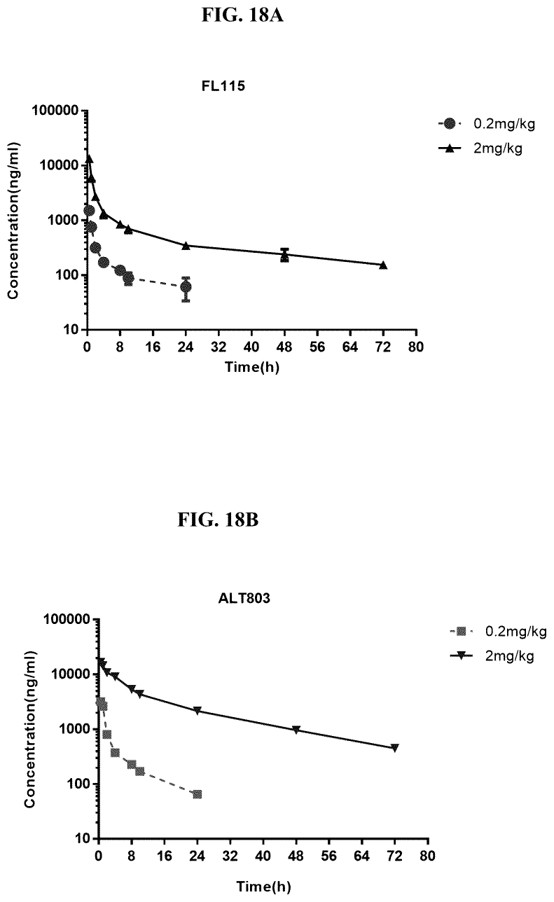

As shown in FIG. 18A and FIG. 18B, the results indicated that there was a consistent trend of change in the serum concentration in the mice of the FL115 group and ALT803 group. Following a single intravenous injection of 0.2 mg/kg FL115 in mice, the serum concentration of FL115 reached the maximum at 30 min after the injection, began to decrease at about 2 hours after the injection, and was essentially stable after 24 hours. The serum half-life (T_{1/2}) of FL115 was 11.11 h. Following a single intravenous injection of 0.2 mg/kg ALT 803 in mice, the serum concentration of ALT 803 reached the maximum at 30 min after the injection, began to decrease at about 2 hours after the injection, and was essentially stable after 72 hours. The serum half-life (T_{1/2}) of ALT 803 was 12.66 h. This revealed that FL115 and ALT803 had similar pharmacokinetic profile.

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains.

The invention illustratively described herein suitably may be practiced in the absence of any element or elements, limitation or limitations which is not specifically disclosed herein.

Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Other embodiments are within the following claims.

We claim:

1. A fusion protein comprising an interleukin-15 (IL-15) receptor α sushi domain fused to an Fc monomer, wherein the IL-15 receptor α sushi domain comprises the amino acid sequence of SEQ ID NO: 5 and the Fc monomer comprises the amino acid sequence of SEQ ID NO: 6.

2. The fusion protein of claim 1, wherein the carboxyl-terminus of the IL-15 receptor α sushi domain is fused to the amino-terminus of the Fc monomer via a linker having the amino acid sequence of SEQ ID NO: 7.

3. A protein complex comprising the fusion protein of claim 1 and an IL-15.

4. The protein complex of claim 3, wherein the IL-15 comprises the amino acid sequence of SEQ ID NO: 3.

5. The protein complex of claim 3, wherein the IL-15 comprises the amino acid sequence of SEQ ID NO: 4.

6. A protein complex comprising the fusion protein of claim 2 and an IL-15.

7. The protein complex of claim 6, wherein the IL-15 comprises the amino acid sequence of SEQ ID NO: 3.

8. The protein complex of claim 6, wherein the IL-15 comprises the amino acid sequence of SEQ ID NO: 4.

9. A pharmaceutical composition comprising the fusion protein of claim 1, a protein complex comprising the fusion protein of claim 1 and an IL-15, a nucleic acid molecule encoding the fusion protein of claim 1 or a host cell comprising the nucleic acid molecule, or one or more nucleic acid molecules encoding the fusion protein of claim 1 and an IL-15 or one or more host cells comprising the one or more nucleic acid molecules, and a pharmaceutically acceptable excipient, diluent, or carrier.

10. A fusion protein comprising the amino acid sequence of SEQ ID NO: 1.

11. A protein complex comprising the fusion protein of claim 10 and an IL-15.

12. The protein complex of claim 11, wherein the IL-15 comprises the amino acid sequence of SEQ ID NO: 3.

13. The protein complex of claim 11, wherein the IL-15 comprises the amino acid sequence of SEQ ID NO: 4.

14. A pharmaceutical composition comprising the fusion protein of claim 10, a protein complex comprising the fusion protein of claim 10 and an IL-15, a nucleic acid molecule encoding the fusion protein of claim 10 or a host cell comprising the nucleic acid molecule, or one or more nucleic acid molecules encoding the fusion protein of claim 10 and an IL-15 or one or more host cells comprising the one or more nucleic acid molecules, and a pharmaceutically acceptable excipient, diluent, or carrier.

15. A fusion protein comprising the amino acid sequence of SEQ ID NO: 2.

16. A protein complex comprising the fusion protein of claim 15 and an IL-15.

17. The protein complex of claim 16, wherein the TL-15 comprises the amino acid sequence of SEQ ID NO: 3.

18. The protein complex of claim 16, wherein the IL-15 comprises the amino acid sequence of SEQ ID NO: 4.

19. A pharmaceutical composition comprising the fusion protein of claim 15, a protein complex comprising the fusion protein of claim 15 and an IL-15, a nucleic acid molecule encoding the fusion protein of claim 15 or a host cell comprising the nucleic acid molecule, or one or more nucleic acid molecules encoding the fusion protein of claim 15 and an IL-15 or one or more host cells comprising the one or more nucleic acid molecules, and a pharmaceutically acceptable excipient, diluent, or carrier.

20. A protein complex comprising an IL-15 having the amino acid sequence of SEQ ID NO: 3, and a fusion protein having an interleukin-15 (IL-15) receptor α sushi domain fused to an Fc monomer, wherein the IL-15 receptor α sushi domain consists of the amino acid sequence of SEQ ID NO: 5, the Fc monomer consists of the amino acid sequence of SEQ ID NO: 6, and the carboxyl-terminus of the IL-15 receptor α sushi domain is fused to the amino-terminus of the Fc monomer via a linker having the amino acid sequence of SEQ ID NO: 7.

21. The protein complex of claim 20, wherein the IL-15 consists of the amino acid sequence of SEQ ID NO: 3.

22. The protein complex of claim 20, wherein the fusion protein consists of the amino acid sequence of SEQ ID NO: 1.

23. The protein complex of claim 20, wherein the fusion protein consists of the amino acid sequence of SEQ ID NO: 2.

24. A pharmaceutical composition, comprising the protein complex of claim 20, one or more nucleic acid molecules encoding the protein complex, or one or more host cells comprising the one or more nucleic acid molecules, and a pharmaceutically acceptable excipient, diluent, or carrier.

25. A nucleic acid molecule encoding the fusion protein of claim 1.

26. A host cell comprising the nucleic acid molecule of claim 25.

27. A method of producing the fusion protein, comprising culturing the host cell of claim 26 under conditions sufficient to express the fusion protein, and isolating the fusion protein.

28. One or more nucleic acid molecules, encoding the fusion protein and the IL-15 of the protein complex of claim 3.

29. One or more host cells comprising the one or more nucleic acid molecules of claim 28.


SEQUENCE LISTING (8 sequences)

SEQ ID NO: 1 - protein, 287 aa, synthetic construct
  source 1..287: note = IL15Ralpha-GGGGS-sFc
ITCPPPMSVEHADIWVKSYSLYSRERYICNSGFKRKAGTSSLTECVLNKATNVAHWTTPSLKCIRGGGGSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVMHEALHNHYTQKSLSLSPGK

SEQ ID NO: 2 - protein, 297 aa, synthetic construct
  source 1..297: note = IL15Ralpha-(GGGGS)3-sFc
ITCPPPMSVEHADIWVKSYSLYSRERYICNSGFKRKAGTSSLTECVLNKATNVAHWTTPSLKCIRGGGGSGGGGSGGGGSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVMHEALHNHYTQKSLSLSPGK

SEQ ID NO: 3 - protein, 114 aa, synthetic construct
  source 1..114: note = IL-15 mutant
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 4 - protein, 114 aa, synthetic construct
  source 1..114: note = WT IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 65 aa, synthetic construct
  source 1..65: note = IL15Ralpha sushi domain
ITCPPPMSVEHADIWVKSYSLYSRERYICNSGFKRKAGTSSLTECVLNKATNVAHWTTPSLKCIR

SEQ ID NO: 6 - protein, 217 aa, synthetic construct
  source 1..217: note = sFc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVMHEALHNHYTQKSLSLSPGK

SEQ ID NO: 7 - protein, 5 aa, synthetic construct
  source 1..5: note = GGGS linker
GGGGS

SEQ ID NO: 8 - protein, 15 aa, synthetic construct
  source 1..15: note = (GGGGS)3 linker
GGGGSGGGGSGGGGS